SEQUENCE LISTING (8 sequences)

SEQ ID NO: 1 - protein, 297 aa, Homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 2 - DNA, 1889 bp, Homo sapiens
  source 1..1889: mol_type = genomic DNA
gcacttggcttcaaagctggctcttggaaattgagcggagagcgacgcggttgttgtagctgccgctgcggccgccgcggaataataagccgggatctaccatacccattgactaactatggaagattataccaaaatagagaaaattggagaaggtacctatggagttgtgtataagggtagacacaaaactacaggtcaagtggtagccatgaaaaaaatcagactagaaagtgaagaggaaggggttcctagtactgcaattcgggaaatttctctattaaaggaacttcgtcatccaaatatagtcagtcttcaggatgtgcttatgcaggattccaggttatatctcatctttgagtttctttccatggatctgaagaaatacttggattctatccctcctggtcagtacatggattcttcacttgttaagagttatttataccaaatcctacaggggattgtgttttgtcactctagaagagttcttcacagagacttaaaacctcaaaatctcttgattgatgacaaaggaacaattaaactggctgattttggccttgccagagcttttggaatacctatcagagtatatacacatgaggtagtaacactctggtacagatctccagaagtattgctggggtcagctcgttactcaactccagttgacatttggagtataggcaccatatttgctgaactagcaactaagaaaccacttttccatggggattcagaaattgatcaactcttcaggattttcagagctttgggcactcccaataatgaagtgtggccagaagtggaatctttacaggactataagaatacatttcccaaatggaaaccaggaagcctagcatcccatgtcaaaaacttggatgaaaatggcttggatttgctctcgaaaatgttaatctatgatccagccaaacgaatttctggcaaaatggcactgaatcatccatattttaatgatttggacaatcagattaagaagatgtagctttctgacaaaaagtttccatatgttatatcaacagatagttgtgtttttattgttaactcttgtctatttttgtcttatatatatttctttgttatcaaacttcagctgtacttcgtcttctaatttcaaaaatataacttaaaaatgtaaatattctatatgaatttaaatataattctgtaaatgtgtgtaggtctcactgtaacaactatttgttactataataaaactataatattgatgtcaggaatcaggaaaaaatttgagttggcttaaatcatctcagtccttatggcagttttattttcctgtagttggaactactaaaatttaggaaaatgctaagttcaagtttcgtaatgctttgaagtatttttatgctctgaatgtttaaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttataggagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagttgcttatcttggctttcgagtctgagtttaaaactacacattttgacatagtgtttattagcagccatctaaaaaggctctaatgtatatttaactaaaattactagctttgggaattaaactgtttaacaaataa

SEQ ID NO: 3 - protein, 398 aa, Homo sapiens
MPRPGHPRPASGPPRLGPWERPTELCLETYDKPPQPPPSRRTRRPDPKDPGHHGPESITFISGSAEPALESPTCCLLWRPWVWEWCRAAFCFRRCRDCLQRCGACVRGCSPCLSTEDSTEGTAEANWAKEHNGVPPSPDRAPPSRRDGQRLKSTMGSSFSYPDVKLKGIPVYPYPRATSPAPDADSCCKEPLADPPPMRHSLPSTFASSPRGSEEYYSFHESDLDLPEMGSGSMSSREIDVLIFKKLTELFSVHQIDELAKCTSDTVFLEKTSKISDLISSITQDYHLDEQDAEGRLVRGIIRISTRKSRARPQTSEGRSTRAAAPTAAAPDSGHETMVGSGLSQDELTVQISQETTADAIARKLRPYGAPGYPASHDSSFQGTDTDSSGAPLLQVYC

SEQ ID NO: 4 - DNA, 1855 bp, Homo sapiens
  source 1..1855: mol_type = genomic DNA
aagtgtgtccctagcggcggcccgtgcagcgctcccgcgagacgctcacctgcgccccaggtgagcggcgagggggcgggggaggggctgagccggaggcggctcacctggcgggacaggtgcctggctgctacaaaccatgcaatgagccatgccccgccctggacacccccgcccagcatctgggcctccacgcttgggaccgtgggagcggccaacagagctatgtctggagacatatgataaaccacctcagcccccaccaagccgccgcacccgtagaccagaccccaaggaccctggccaccatgggccagagagcattaccttcatctctggctctgctgagccggcccttgagtcccccacctgctgcctgctctggcgaccctgggtgtgggagtggtgccgggctgccttctgcttccgccgctgccgggattgcctccagcgctgtggagcctgtgtgcggggatgcagcccctgcctgtctactgaggactccactgaggggactgctgaagccaactgggccaaggagcacaatggagtgccccccagccctgatcgtgcaccccccagccggcgggatggccagcggctcaagtcaaccatgggcagcagcttcagctaccccgatgttaagctcaaaggcatccctgtgtatccctacccgagggccacctccccagcccctgatgcggactcctgctgcaaggagccactggccgatcccccacccatgcgacacagcctgcccagcacctttgccagtagtcctcgtggctccgaggagtactattctttccatgagtcggacctggacctgccggagatgggcagtggctccatgtcgagccgagaaattgatgtgctcatcttcaagaagctgacagagctgttcagcgtacaccagatcgatgagctggccaagtgcacatcagacactgtgttcctggagaagaccagtaagatctcggaccttatcagcagcatcacgcaggactaccacctggatgagcaggatgctgagggccgcctggtacgcggcatcattcgcattagtacccgaaagagccgtgctcgcccacagacctcggagggtcgttcaactcgggctgctgccccaaccgctgctgcccctgacagtggccatgagaccatggtgggctcaggtctcagccaggatgagctgacagtgcagatctcccaggagacgactgcagatgccatcgcccggaagctgaggccttatggagctccagggtacccagcaagccatgactcatccttccagggcaccgacacagactcgtcgggggcacccttgctccaggtgtactgctaacccctgccaggcccagctgccacaccctttctgggagaagcatggcctacagaatgaagagggggaccaggaacccctgtgggagaggcttagacctgaagcagtgcccactctggctcctcctgccttggctgactgggttcctggaccatgtgcatttcactgggccatgggatctacatctccttgcatccccagctggtctgatccctgccagggccccttccttcctgctcatggtcttcaggtggcctgatcatggaaagtaaggagttaggcattaccttctgggagtgaaccctgactccatccccctattgccaccctaaccaatcatgcaaacttctccctccctggggtaattcaacagttaaaagaagcttatcttaaatgtattgtattggggggtgggcagggcccactctatgttatgttaaggagttggttctggttcttggctgatgttctgtatcttaacatgaccacagtttgtaagtacaaaggtaa

SEQ ID NO: 5 - protein, 508 aa, Homo sapiens
MIYEESKMNLEQERPFVCSAPGCSQRFPTEDHLMIHRHKHEMTLKFPSIKTDNMLSDQTPTPTRFLKNCEEVGLFSELDCSLEHEFRKAQEEESSKRNISMHNAVGGAMTGPGTHQLSSARLPNHDTNVVIQQAMPSPQSSSVITQAPSTNRQIGPVPGSLSSLLHLHNRQRQPMPASMPGTLPNPTMPGSSAVLMPMERQMSVNSSIMGMQGPNLSNPCASPQVQPMHSEAKMRLKAALTHHPAAMSNGNMNTMGHMMEMMGSRQDQTPHHHMHSHPHQHQTLPPHHPYPHQHQHPAHHPHPQPHHQQNHPHHHSHSHLHAHPAHHQTSPHPPLHTGNQAQVSPATQQMQPTQTIQPPQPTGGRRRRVVDEDPDERRRKFLERNRAAATRCRQKRKVWVMSLEKKAEELTQTNMQLQNEVSMLKNEVAQLKQLLLTHKDCPITAMQKESQGYLSPESSPPASPVPACSQQQVIQHNTITTSSSVSEVVGSSTLSQLTTHRTDLNPIL

SEQ ID NO: 6 - DNA, 8539 bp, Homo sapiens
  source 1..8539: mol_type = genomic DNA
gttcttcaacatttacaacaaagttgattctgtgtagggttggaggctagacagttccacaaatttttagtcacattttccatgtcagttaaatctagggagttcaagactactggaaaaattagtctcattactaaaagaaacttagagaacgagggaggtaccagagtctaggaggtacctctgggttgcagaagtaattgtaaaataccagacctgttctttttactaaaagctagtttcactatcttctggtctgaaatactgaggcaaatactcaagacttattttcttcctaatcttgctggtgaaacagaagttactagaaagaaaggaagaaaaaacttgatttggtgactgcaggaagcaacacgttgctgcttttattctacagataatgatttatgaggaatccaagatgaatttggagcaggagaggccgtttgtctgcagtgccccaggctgctcccagcgcttcccaacagaggaccatctgatgattcataggcacaaacatgaaatgactttgaagtttccttcaataaaaacagacaatatgttatcagatcaaactccgaccccaacgagattcctgaagaactgcgaggaggtgggcctcttcagcgagctggactgctccctggagcacgagttcaggaaggctcaggaagaggagagcagcaagcggaatatctcgatgcataatgcagttggtggggccatgacggggcccggaactcaccagcttagcagcgctcggctgcccaaccatgacaccaacgttgtgattcagcaagccatgccgtcgcctcagtccagctctgtcatcactcaggcaccttccaccaaccgccagatcgggcctgtcccaggctctctatcttctctgctacatctccacaacagacagagacagcccatgccagcctccatgcctgggaccctgcccaaccctacaatgccaggatcttccgccgtcttgatgccaatggagcgacaaatgtcagtgaactccagcatcatggggatgcaaggtccaaatctcagcaacccctgtgcttctccccaggtccagccaatgcattcagaagccaaaatgaggttgaaggctgcattgactcaccaccctgctgccatgtcaaatgggaacatgaacaccatgggacacatgatggagatgatgggctcccggcaggaccagacgccacaccatcacatgcactcgcacccgcatcagcaccagacactgccaccccatcacccttacccacaccagcaccagcacccagcacaccatcctcaccctcaaccccatcaccagcagaaccatccacatcaccactcccattcccaccttcatgcacacccagcacatcaccagacctcgccacatccgcccctgcacaccggcaaccaagcacaggtttcaccagcaacacaacagatgcagccaacccagacaatacagccaccccagcccacaggggggcgccggcgaagggtggtagacgaggatccggacgagaggcggcggaaatttctggaacggaaccgggcagctgccacccgctgcagacagaagaggaaggtctgggtgatgtcattggaaaagaaagcagaagaactcacccagacaaacatgcagcttcagaatgaagtgtctatgttgaaaaatgaggtggcccagctgaaacagttgttgttaacacataaagactgcccaataacagccatgcagaaagaatcacaaggatatctaagtccagagagtagccctcctgctagtcctgtcccagcttgctcccagcaacaagtcatccagcataataccatcactacttcctcatcggtcagcgaggtggtaggaagctccaccctcagccagctcaccactcacagaacagacctgaatccgattctttaaaatgcaccatcagacctggcctccaagaagagctgtagcgtaccatgcgtcctttcttttaagggcatttttagaattaactcagacctggaagactcctcagttcttcaaagactggctttcatttttatagttattatggaaatgttgtcttttatacttagttatataagaaaaaagggagttatgcaattaatatctatcagcttgggaaacgctttggtgcttttctccagttttctggtaccagttacttgtttataaactgaaccttttctgtatatagccatggtttcattcttatcagtccaaccctttgcctgaaacattgaatcttgttaaaccacagcttttagctaaaatgaggtatacctagatgtcaagtaagacagatccaaggtaactgggtaggaaatcttttgacatcttaactcatgttgagtttgtgctgtggtgtcaccagaattccagataaacacacagcctttcccatacctttttttttcttactataaaatattataagatccattgatgtccaaataataccaccaagcatctcttcacctctcctcctcttggtccacttgctaatgcccagttttcttctccatttccactttttcttaggctccctatttactattcattttgacttccttctgttttatttttttccctttagcattgcatgtgaataagaaaataatgtttaaagaaaaaaaaaaaaaagcaaacctccaaaacgtggacctaaccattgcttcacttacacttcacccacagctggagttcattcaactcttgcttttcacaaaatagtaaccaggagatgtttaatgtgcctgatttaatgtttttaataatcacagcaaatgaaaggtggtttagttataagtgaagcatggttgaataccagctggggagacactagggaagggagctttgtaagccttgattgcgaaagtccaaattttgatgtggggctataacatgacacccttggattgcgactggttttatacggcctgcctataacgttgaaaatccatgtactacataataattcagaagggctctattcactacacagattacattgttcaatcatcagctgctaatagcctaagatttatttttttttttttcttaagcctatggaaccggctttgctgttctggggggtgaaaatagactaactactggagaaacaaagagagaaagaaaacccagtgtttccataggggcacttttagccttcccacaacagttaagcactctttgactgctgaaggaaccccatggatgaggtgcaggctacttcactctttttttttcttttttgagacagagtctcacctattgcccagactgaagtgcagtggtgcgatcatggatcactgcagcagcatcctccgagttcaagctatccttccacctccgcctcctgagtagctgggaccacaggttcacataaccatgcctggctaatttatttttacttttattttaaaataaaagatgaggtctgtcttatgttgcccaggctggtctcaaactatcctacttcttcctcccaaagtgttgggattataggtgtgagccactgcacccagcctacttcactcttctgaattattctgatttattttcaacaacttttgtgaacttgcccgtgatacaaagcagatagtccctgaaccacagtcgtgcctccttgaaacaagccattctactgtgctaatgttttaatatcacatctcacaaataacaggggtgaatgtttctctctagcaatctaggcaggtgctggtgtttcatctccatttgaatgcttgacctcttaatgtgtgtgtgtgtgtgtgtgtgtgtgtgtgtgttcatgggttttaaaagaacagtattttacaaaaggtgtagcttttataagagtgcagaaaagggaaggatgtgtttttttctctcactatagtataagaatctattttggagaaaaaaagaaaatatgagggtctcgaagcatgatttttatataactagtttcagttttatctaataacttactttttaaatcaatatttatcaacaatctttccttgtatgcagtgctttcaaaagatggttttgagtgtccagtgaaacttatgacttggatatatggttgaagaatcaaaacaaaagcaaaaaaaaaaagcaaaaaaagaaaagagaaaaaaagaaaaaatgcaaatggaataattttctattatattttagacaaacatatcattttcgagtattttaaatactgaattcatagttgttgttttttaaattccaacagtaacagctgaatggtttaatctgactggcttcctaagaaatgtttaagactcagctttaaaaagaagttaacattcatatctctgttttgaaatcaaaaatcatatttcaaaattctttcctaggaccatctatgtgtctcccctcccctccacaaaaaggagaaagagtgcattaaaatgtttagttgggttttttaatttttaatttttatgttatgttttgctttgttttaagtaaacaaaaatttttctttctttactgcatgcatagcacttaataaaatggatttttaaaaaatccactagtaatatcagaatgtccagggagtgactgtcactacaatgatggtttagtttacttctgttccaccttttgattgaaatatttagttgttaggctgaaagcctcggcagttaagaacttgcctgagttttcttcgttcagcaacttgacagtttgactgatgtgcattatatatagctcaattatgtctgttttttatgctaagtaggaaaaccaaccacacacattagcaaaccggcctcaacatataattagaataaactgtcttcttgttctactcagggcctttaggtgtgttcattcacggtatggaaatacagtaaatgaaagattccaactagttgtcagtgcttcttgaaattccaaacagaaagatacattggtcaaatccaacacttggcttatcaatattaagtcttttacctaaaggcccagccgtcaccagacaacagaataatcaatctgcctgaaaatccctcctccttgtcctacactttttgcctgtttgggagaatatctttgtactccattctcctccctcagccagttactgggtcacccatccatgtgttcatgaatcaatcatcacggcctgcagagcacctgtcctaaggagggaaaatcctgtcacactgcctctccccattcgtgtgtggttttcttgatcggtgagatctgtctctgaagtcactgccagcctccctgggaacgtctatagtgcctcccctgccttatgtgatgggagttaacaactcagataagtacacctgagagcatttctatcaggtaaactgtcacttaaatggaggtgtccacatcttaattgtttctccttgacacatttctcaatccacgaagccaggagaggtagagtgaaaatcccagccatggatgaatgtactaatttgaaagccaagtgttaagtcggatgttttcccgttacactactactcagccctctcctgcggccacatcaacggatgcaagtcacagtcttaacacagcctgtgggagacaagcagtttgtgtgctcacagtatatattatagtaattagggtgacttagagcaaatactcttcagatcctatgtagtcagtgaaacaaaatggagagcgtattctgatagaaggacgtcgacggtgaatgttctggtggttgttgcctgttaagtaaactttagtgtgtaagttgagtttgtcattaaaatcataaaccagctgcggtaacagacaagcctttggctggggagttttaagcctcggtaactgctataaaactagccatccagttaggatagaatgtgtttctttctggttaaaaaaaggaaaaaccatctaagaaaatatatatgtatgtatgtgtgtatacagtggaattcaaaggaccaaagcaaaatttgaacaggaatctattaatttagaattttataagatatttattaataaatgttatttttaaacattccatttgaacagtattctgtaggatctacttgtttttaaagtgttagtccataataaactactatagttatgtgtattttcatttttcagggtttcaaatggctattctccatcatttggtggaaatgtttgcttagatctctgtgcatagacatttcaaggatttttattgctctgtgagttattttttaatcaacattctgaacagttttttttaaacatttatttctgtgtgttcatttttaaagtaagctctttcatttaggaagcagagttcagctaaagggaatcagtaactctaactggaacagctttcttgtagaagtgtaaaaacagcttcatctctgcctctctccaccccaccccaatttcctagaaagccttgcactattcagctcccttagtgctttttgtcccttcccgaacaatatgcagtagctttaagccattcaagctccattatgcagtatatctgagaagggaaaggaaacaacccatttaaatttgaataaaaccgtgcctatgcgaacagtagcaatttagaatctcttttctgcttttaaaataatttatatttaaaaattgcactttagctttttgatccctttgtatttctcttattctctttctaacctcttctctgtcctcaaacttgcctttgctctcctttacaataccccccacccctcctccaaggctctgagcggcatcatttaaaatactttacagatatttgcaccaggtacatttatgtgcgtccattggtagcacagctgagacctgtgtctcacatcagcctaggtgaagcctactacaagaatgccaaggagaagagccagtacactatatggtttatactctttatccctttattcatagcatgttttttaaaaatgttatattatgcaacagatgtgaggcagcagctaagctatacttaagaattttctctcaccttccaaaccaaagtgtcctgaataagccaggagacttattcttttgtgcaccctggtgcacatctgactgttgtcctagccatagactctctgaggccactgaaagaacagtggccctatcgatttcattcctaggtctcaaaaatacaatgttgccttgtaacataattagggacagcacctctatttcacaattataatctaaggtaggataagacgacacagcagcaataaacttacaagtaaaattcaataccaaaacaaacacaaagaaatttaaaaaacaaaaaacctagctcatcatgttgtgaaaatgaaaaagtgaatgtccattcaaaatattttactatttcttgtggagtttttcagtgatgtaatgcttgtagccaaattgcttaaagagtgtttatatatttttttccttataaattgtctattttttaaaaaagctatttaaccacagctgaagtggggggtaaggccaaattgccaacacttgttaaaagattaatactcttaagtggcactctgatacctttccaacttgtcatcagaaaggaatcaataattaccaactgttgtatttagaccaacttacaatatctagctcattagaagccaggatctagaaagctccttctaagccatttaagatattcttacattgagcttcatattatagaactttataggattggatattttacaatagaataatttagcctcaggactgagaatgtggaagctgaataaattagctttaaatacatcattaaaatcttatgcacaataagctcattagattctagttttctcctttagaataccaatgccacagacactacaggagataatgaaaggtatcagttgtgttgagtggagggagtttaagagaaaggacccttcccaaccagcagccagtagaaaatacaacctactcacctttttcccttctaagttctgctaaatcacatctgcctcatagagaaaggaatgttgcctttgagaactgtcttggagaacagataagcttgaaatgttctctctagagaggacatagggtttgggatcctctgaaaaggcccagaaaaatagctcagttcaaatacaatgttctaggacaattggaatataaatattgtccaaaaatataattaaaagaaaaaagtttagcactgtgtaaagtaagtgttaactgaggaagtcccaaaaaggtgctgtcactttaagttctggacttggggttctttgtatttgtaaacagcaaagcatttgtgtttgtttgtctatttgtaaagcaaccaccttccttattggaaggagaaaaaaaggggtacatacatgtaaatacttgctgcagcatttaatatgtttaattttgtgttaagctttttgttgcatcgtgaacacatttattgttaccaatggacaatgagttcattaagactgttcaactaggtcagatttttacatctctttctagcaagaagagacaagattttgtgcatttgtacaaatgttaatatcactgcaattccaatataataaagcactcaaatgcaaataa

SEQ ID NO: 7 - protein, 125 aa, Homo sapiens
MNEVSVIKEGWLHKRGEYIKTWRPRYFLLKSDGSFIGYKERPEAPDQTLPPLNNFSVAAPCCLFPLPAECQLMKTERPRPNTFVIRCLQWTTVIERTFHVDSPDEREEWMRAIQMVANSLKQRAP

SEQ ID NO: 8 - DNA, 546 bp, Homo sapiens
  source 1..546: mol_type = genomic DNA
aaacacaaggaaagggaaccagcgcagcgtggcgatgggcgggggtagagccccgccggagaggctgggcggctgccggtgacagactgtgccctgtccacggtgcctcctgcatgtcctgctgccctgagctgtcccgagctaggtgacagcgtaccacgctgccaccatgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagccccctgctgtctgttccctctgcctgcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccagg

(19) United States
(12) Patent Application Publication
(10) Pub. No.: US 2026/0250772 A1
(43) Pub. Date: Aug. 27, 2026

(54) BIOMARKER FOR PREDICTING LIKELIHOOD OF DEVELOPING GASTRIC CANCER AND USE THEREOF

(71) Applicant: SEOUL NATIONAL UNIVERSITY HOSPITAL, Seoul (KR)

(72) Inventors: Soo Jeong CHO, Seoul (KR); Sang Gyun KIM, Seoul (KR); Miree PARK, Gwangmyeong-si, Gyeonggi-do (KR)

(73) Assignee: SEOUL NATIONAL UNIVERSITY HOSPITAL, Seoul (KR)

(21) Appl. No.: 18/870,119

(22) PCT Filed: Sep. 27, 2022
(86) PCT No.: PCT/KR2022/014463

§ 371 Date: Nov. 27, 2024

(30) Foreign Application Priority Data
May 31, 2022 (KR) 10-2022-0067245
Sep. 20, 2022 (KR) 10-2022-0118936

(51) Int. Cl. C12Q 1/6886; G01N 33/5753

(52) U.S. Cl. CPC C12Q 1/6886; G01N 33/5753; C12Q 2600/118; C12Q 2600/158

(57) ABSTRACT

Provided are a method for predicting likelihood of developing gastric cancer in a subject, including manufacturing a preparation for measuring the level of mRNA of the genes of CDK1, KDF1, CREB5, and/or AKT2, or the proteins expressed therefrom, measuring with the preparation the level of mRNA of the genes or proteins in a biological sample isolated from the subject, comparing the level of mRNA or protein to that from a control group, and optionally determining that the risk of developing gastric cancer is high when the level of the mRNA or protein in the subject is higher than that from the control group; and a method for predicting prognosis of early gastric cancer patients after treatment by endoscopic submucosal dissection, including the same steps, where the methods have high accuracy and enable the prediction via a one-time measurement by multi-plex PCR with a single tissue sample.

### TECHNICAL FIELD

The present invention relates to a biomarker for predicting the likelihood of developing gastric cancer and use thereof.

The present invention claims priority based on Korean Patent Application No. 10-2022-0067245 filed on May 31, 2022, and Korean Patent Application No. 10-2022-0118936 filed on Sep. 20, 2022. All contents disclosed in the specifications and drawings of these applications are incorporated herein by reference.

### BACKGROUND ART

Regarding gastric cancer, significant progress has been made not only in surgical procedure and adjuvant chemotherapy but also in early diagnosis methods. However, gastric cancer remains the second leading cause of cancer-related deaths worldwide, regardless of gender, and continues to be one of the most common causes of death. However, the exact mechanism underlying the most important gastric cancer development and tumor progression has not yet been fully elucidated. In particular, it is known that the reason why Korea has the highest incidence of gastric cancer in the world is because underlying gastritis has progressed significantly.

Metachronous Gastric Cancer (MGC) refers to cases where gastric cancer is diagnosed more than 12 months after the diagnosis, and specifically, an example is gastric cancer that newly develops in a site other than the original treatment site during follow-up after gastric cancer treatment with endoscopic resection.

In patients with early gastric cancer treated with endoscopic submucosal dissection (ESD), the probability of developing metachronous gastric cancer in the normal gastric mucosa other than the area where the cured gastric cancer is resected is very high, with an annual incidence of 3-5%, and the risk of developing it is about 100 times higher than that of normal people. Therefore, predicting the occurrence of such metachronous gastric cancer is very important for predicting the prognosis of patients.

However, since there is no predictor of metachronous gastric cancer after endoscopic resection in gastric cancer, endoscopy is performed once a year for life in all patients, but there is a lot of f/u loss in the middle.

In conventional endoscopy, underlying gastritis (intestinal metaplasia, atrophic gastritis) has been used as a predictor of future gastric cancer development, but inter-observer or intra-observer variability is severe. To overcome this, OLGA (Operative Link on Gastritis Assessment) and OLGIM (Operative Link on Gastritis Assessment based on Intestinal Metaplasia) stages have been developed. However, these require at least 2 biopsies from 5 locations in the endoscopy, requiring a total of 10 endoscopic tissue specimens. Accordingly, the risk of complications such as bleeding increases, so they are rarely used in actual clinical practice.

In addition, there is Korean Patent Registration Publication No. 10-2253304 regarding a specific marker for gastric cancer recurrence, but the biomarker used for diagnosing or predicting metachronous gastric cancer according to the present invention has not been disclosed.

### DISCLOSURE

### Technical Problem

Against this background, the inventor of the present invention confirmed that the expression levels of CDK1 and KDF1 measured in normal mucosa other than gastric cancer at the time of performing endoscopic submucosal dissection for early gastric cancer can be used to predict the occurrence of metachronous gastric cancer in the future, and confirmed the correlation between the expression of CREB5 and AKT2 isoform genes and OLGIM stage, thereby confirming that it is possible to predict not only metachronous gastric cancer but also the occurrence of gastric cancer in normal people through a single gene expression measurement with a single tissue specimen, thereby completing the present invention.

The present invention provides a personalized approach to the observation of future metachronous gastric cancer occurrence through risk stratification in patients who underwent endoscopic submucosal dissection for early gastric cancer through the expression patterns of CDK1, KDF1, CREB5, and AKT2 isoform genes, and also provides a quantitative method for predicting gastric cancer occurrence in normal people, as well as a method for predicting gastric cancer occurrence in normal people with a family history of gastric cancer or risk factors.

Therefore, an object of the present invention is to provide a composition for predicting the likelihood of developing gastric cancer, comprising a preparation for measuring the level of mRNA of any one or more genes selected from the group consisting of CREB5 and AKT2, or the level of proteins expressed therefrom.

Another object of the present invention is to provide a kit for predicting the likelihood of developing gastric cancer comprising the composition.

Another objective of the present invention is to provide a composition for predicting the likelihood of developing metachronous gastric cancer or a composition for predicting the prognosis of early gastric cancer patients treated with endoscopic submucosal dissection, comprising a preparation for measuring the level of mRNA of any one or more genes selected from the group consisting of CDK1, KDF1, CREB5, and AKT2, or the level of proteins expressed therefrom.

Another object of the present invention is to provide a method for providing information for predicting the likelihood of developing gastric cancer, comprising measuring the level of mRNA of any one or more genes selected from the group consisting of CREB5 and AKT2, or the level of proteins expressed therefrom.

Another objective of the present invention is to provide a method for providing information for predicting the likelihood of developing metachronous gastric cancer or a method for providing information for predicting the prognosis of early gastric cancer patients after treatment by endoscopic submucosal dissection, comprising measuring the level of mRNA of any one or more genes selected from the group consisting of CDK1, KDF1, CREB5, and AKT2, or the level of proteins expressed therefrom.

### Technical Solution

To achieve the above objectives, the present invention provides a composition for predicting the likelihood of developing gastric cancer, comprising a preparation for measuring the level of mRNA of any one or more genes selected from the group consisting of CREB5 and AKT2, or the level of proteins expressed therefrom.

In one embodiment of the present invention, the gastric cancer may comprise metachronous gastric cancer, but is not limited thereto.

In another embodiment of the present invention, the composition may further comprise a preparation for measuring the level of mRNA of any one or more genes selected from the group consisting of CDK1 and KDF1, or the level of proteins expressed therefrom, but is not limited thereto.

In yet another embodiment of the present invention, the preparation for measuring the mRNA level of the gene may be a primer or a probe that specifically binds to the gene or mRNA, but is not limited thereto.

In yet another embodiment of the present invention, the preparation for measuring the protein level may be an antibody or an aptamer that is specific to the protein, but is not limited thereto.

In addition, the present invention provides a kit for predicting the likelihood of developing gastric cancer, comprising the composition.

In one embodiment of the present invention, the kit may be one or more selected from the group consisting of a reverse transcription polymerase chain reaction (RT-PCR) kit, a real-time polymerase chain reaction (qRT-PCR) kit, a DNA chip kit, an Enzyme-linked immersive assay (ELISA) kit, and a protein chip kit, but is not limited thereto.

In addition, the present invention provides a composition for predicting the likelihood of developing metachronous gastric cancer, comprising a preparation for measuring the level of mRNA of any one or more genes selected from the group consisting of CDK1, KDF1, CREB5, and AKT2, or the level of proteins expressed therefrom.

In addition, the present invention provides a composition for predicting the prognosis of early gastric cancer patients treated by endoscopic submucosal dissection, comprising a preparation for measuring the level of mRNA of any one or more genes selected from the group consisting of CDK1, KDF1, CREB5, and AKT2, or the level of proteins expressed therefrom.

In addition, the present invention provides a method for providing information for predicting the likelihood of developing gastric cancer, comprising: (a) measuring the level of mRNA of any one or more genes selected from the group consisting of CREB5 and AKT2, or the level of proteins expressed therefrom in a biological sample isolated from a subject; and
(b) comparing the measured mRNA or protein level to the level measured in a biological sample isolated from a control group.

In one embodiment of the present invention, the gastric cancer may comprise metachronous gastric cancer, and the method may further comprise measuring the level of mRNA of any one or more genes selected from the group consisting of CDK1 and KDF1, or the level of proteins expressed therefrom in a biological sample isolated from the subject, and comparing the level to that measured in a sample isolated from a control group, but is not limited thereto.

In another embodiment of the present invention, the level of mRNA of the gene may be measured by one or more methods selected from the group consisting of reverse transcription polymerase chain reaction (RT-PCR), competitive reverse transcription polymerase chain reaction (competitive RT-PCR), real-time quantitative reverse transcription polymerase chain reaction (real-time quantitative RT-PCR), multiplex reverse transcription polymerase chain reaction (Multi-plex PCR), real-time polymerase chain reaction (qRT-PCR), RNase protection method, Northern blotting, DNA chip technology assay, methylated DNA binding domain sequencing (MBD-seq), and reduced representation bisulfite sequencing (RRBS), but is not limited thereto.

In yet another embodiment of the present invention, the level of the protein may be measured by one or more methods selected from the group consisting of western blot, enzyme-linked immunosorbent assay (ELISA), radioimmunoassay (RIA), radioimmunodiffusion, ouchterlony immunodiffusion, rocket immunoelectrophoresis, immunohistochemistry (IHC), immunoprecipitation assay, complement fixation assay, fluorescence-activated cell sorting (FACS), and protein chip, but is not limited thereto.

In yet another embodiment of the present invention, the measurement of the mRNA level or protein level of the gene may be measured using a single biological sample, but is not limited thereto.

In yet another embodiment of the present invention, the biological sample may be one or more selected from the group consisting of tissue, cells, whole blood, serum, plasma, saliva, sputum, cerebrospinal fluid, urine, and feces isolated from the subject, but is not limited thereto.

In yet another embodiment of the present invention, the method may further comprise (c) determining that the risk of developing gastric cancer is high when the level of the mRNA or protein measured in the biological sample isolated from the subject is higher than the level measured in a biological sample isolated from the control group, but is not limited thereto.

In addition, the present invention provides a method for providing information for predicting the likelihood of developing metachronous gastric cancer, comprising: (a) measuring the level of mRNA of any one or more genes selected from the group consisting of CDK1, KDF1, CREB5, and AKT2, or the level of proteins expressed therefrom in a biological sample isolated from a subject; and
(b) comparing the measured mRNA or protein level to the level measured in a biological sample isolated from a control group.

In addition, the present invention provides a method for providing information for predicting the prognosis of early gastric cancer patients after treatment by endoscopic submucosal dissection, comprising: (a) measuring the level of mRNA of any one or more genes selected from the group consisting of CDK1, KDF1, CREB5, and AKT2, or the level of proteins expressed therefrom in a biological sample isolated from early gastric cancer patients; and
(b) comparing the measured mRNA or protein level to the level measured in a biological sample isolated from a control group.

In addition, the present invention provides a method for predicting the likelihood of developing gastric cancer, comprising: (a) measuring the level of mRNA of any one or more genes selected from the group consisting of CREB5 and AKT2, or the level of proteins expressed therefrom in a biological sample isolated from a subject; and
(b) comparing the measured mRNA or protein level to the level measured in a biological sample isolated from a control group.

In addition, the present invention provides a method for predicting the likelihood of developing metachronous gastric cancer, comprising: (a) measuring the level of mRNA of any one or more genes selected from the group consisting of CDK1, KDF1, CREB5, and AKT2, or the level of proteins expressed therefrom in a biological sample isolated from a subject; and
(b) comparing the measured mRNA or protein level to the level measured in a biological sample isolated from a control group.

In addition, the present invention provides a method for predicting the prognosis of early gastric cancer patients after treatment by endoscopic submucosal dissection, comprising: (a) measuring the level of mRNA of any one or more genes selected from the group consisting of CDK1, KDF1, CREB5, and AKT2, or the level of proteins expressed therefrom in a biological sample isolated from early gastric cancer patients; and
(b) comparing the measured mRNA or protein level to the level measured in a biological sample isolated from a control group.

In addition, the present invention provides a use of a composition comprising a preparation for measuring the level of mRNA of any one or more genes selected from the group consisting of CREB5 and AKT2, or the level of proteins expressed therefrom for predicting the likelihood of developing gastric cancer.

In addition, the present invention provides a use of a preparation for measuring the level of mRNA of any one or more genes selected from the group consisting of CREB5 and AKT2, or the level of proteins expressed therefrom for manufacturing a preparation for predicting the likelihood of developing gastric cancer.

In addition, the present invention provides a use of a composition comprising a preparation for measuring the level of mRNA of any one or more genes selected from the group consisting of CDK1, KDF1, CREB5, and AKT2, or the level of proteins expressed therefrom for predicting the likelihood of developing metachronous gastric cancer.

In addition, the present invention provides a use of a preparation for measuring the level of mRNA of any one or more genes selected from the group consisting of CDK1, KDF1, CREB5, and AKT2, or the level of proteins expressed therefrom for manufacturing a preparation for predicting the likelihood of developing metachronous gastric cancer.

In addition, the present invention provides a use of a composition comprising a preparation for measuring the level of mRNA of any one or more genes selected from the group consisting of CDK1, KDF1, CREB5, and AKT2, or the level of proteins expressed therefrom for predicting the prognosis of early gastric cancer patients treated by endoscopic submucosal dissection.

In addition, the present invention provides a use of a preparation for measuring the level of mRNA of any one or more genes selected from the group consisting of CDK1, KDF1, CREB5, and AKT2, or the level of proteins expressed therefrom for manufacturing a preparation for predicting the prognosis of early gastric cancer patients treated by endoscopic submucosal dissection.

### Advantageous Effects

The composition for predicting the likelihood of developing gastric cancer according to the present invention not only has high accuracy in predicting the development of gastric cancer, but also has high usability because it can predict gastric cancer with a single measurement method by using multiplex PCR, etc. with a single tissue sample. In addition, it includes a method for quantifying basal gastritis as a quantitative method for predicting the development of gastric cancer as well as metachronous gastric cancer, so it provides a method for predicting the risk of developing gastric cancer even in normal people with a family history of gastric cancer or risk factors. In addition, it has the effect of predicting the prognosis of patients by predicting the development of metachronous gastric cancer in early gastric cancer patients treated with endoscopic submucosal dissection.

### DESCRIPTION OF DRAWINGS

FIGS. 1A to 1E show the results of DESeq2 analysis of normal gastric mucosal tissue around the tumor, that show respectively, the results of correlation analysis according to gene expression level and number of gastric cancer, number of adenomas, the degree of atrophic gastritis (OLGA stage), and the degree of intestinal metaplasia (OLGMI stage) (FIG. 1A), among the candidate genes confirmed to have high expression in the metachronous gastric cancer group, the result showed CDK1 expression level (FIG. 1B), as a result of checking the self-organizing map (SOM) (FIG. 1C), alternative splicing analysis results (FIG. 1D), and the cDNA sequence and amino acid sequence of AKT2 isoform set as the target (FIG. 1E).

FIG. 2 shows the result of selecting a candidate gene as a biomarker for metachronous gastric cancer in consideration of the results of bulk RNA sequencing analysis performed with normal gastric mucosa tissue.

FIG. 3 shows the results of evaluating cell viability in gastric cancer cell lines treated with siRNA.

FIG. 4A shows the results of evaluating validation of CDK1, KDF1, POLR2L, CREB5, and AKT2-isoform gene expression.

FIG. 4B shows the results of western blot confirming the expression levels of CDK1 and KDF1 proteins in normal gastric mucosa tissue around the tumor (control) and patient tissue with metachronous gastric cancer (mGC).

FIG. 4C shows the results of immunohistochemistry confirming the expression level of CDK1 protein in normal gastric mucosa tissue around the tumor (control) and patient tissues with metachronous gastric cancer (mGC).

FIG. 4D shows the results of immunohistochemistry confirming the expression level of KDF1 protein in normal gastric mucosa tissue around the tumor (control) and patient tissues with metachronous gastric cancer (mGC).

FIGS. 5A to 5D show the results of ROC curve analysis of CDK1, KDF1, CREB5, and AKT2-isoform genes and gastric cancer development prediction.

FIG. 6 shows the results showing the expression of the CREB5 gene according to the OLGIM stage.

FIG. 7 shows the results showing the expression of the AKT2 gene according to the OLGIM stage.

### BEST MODE

In one embodiment of the present invention, differentially expressed genes (DEGs) were identified between patients who developed metachronous gastric cancer during follow-up and those who did not, in patients with early gastric cancer treated by endoscopic submucosal dissection whose normal gastric mucosa tissues were obtained and stored at the time of diagnosis. In addition, correlation analysis according to the amount of gene expression and number of metachronous gastric cancer, number of adenomas, the degree of atrophic gastritis (OLGA stage), and the degree of intestinal metaplasia (OLGMI stage), self-organizing map (SOM) analysis, and isoform analysis were performed thereby selecting CDK1, KDF1, ERCC6L, KLK8, MCM2, POLR2L, PTN, CREB5, and AKT2 isoforms as candidate genes (see Example 1).

In another embodiment of the present invention, the differences in gene expression in various gastric cancer cell lines and normal gastric mucosa cell lines were confirmed through qRT-PCR for the candidate genes, and the inhibition of viability in siRNA-treated gastric cancer cell lines was evaluated, thereby selecting CDK1, KDF1, CREB5, and AKT2-isoform as the final target genes (see Example 2-1). It was confirmed through Western blot and immunohistochemistry that the protein expression of CDK1 and KDF1 was higher in patient tissues with metachronous gastric cancer than in patient tissues without metachronous gastric cancer (see Examples 2-2 and 2-3).

In yet another embodiment of the present invention, the validity of the CDK1, KDF1, CREB5 and AKT2-isoform genes was evaluated, and as a result, it was confirmed that the accuracy of each gene marker for predicting metachronous gastric cancer was high, and in particular, the accuracy increased the most when the CDK1, KDF1, and AKT2-isoform markers were combined. In addition, it was confirmed that CREB5 and AKT2 isoforms showed expression differences according to the OLGIM stage, and thus it was confirmed that gastric cancer prediction is possible not only in metachronous gastric cancer but also in normal people with a family history of gastric cancer or risk factors (see Example 3).

Hereinafter, the present invention will be described in detail.

The present invention provides a composition for predicting the likelihood of developing gastric cancer, comprising a preparation for measuring the level of mRNA of any one or more genes selected from the group consisting of CREB5 and AKT2, or the level of proteins expressed therefrom.

In the present invention, the composition may further comprise a preparation for measuring the level of mRNA of any one or more genes selected from the group consisting of CDK1 and KDF1, or the level of proteins expressed therefrom, but is not limited thereto.

In addition, the present invention provides a composition for predicting the likelihood of developing metachronous gastric cancer, comprising a preparation for measuring the level of mRNA of any one or more genes selected from the group consisting of CDK1, KDF1, CREB5, and AKT2, or the level of proteins expressed therefrom.

The composition may comprise one gene or a combination of two, three, or four genes among the four genes.

For example, the gene may be CDK1; KDF1; CREB5; AKT2; CDK1 and KDF1; CDK1 and CREB5; CDK1 and AKT2; KDF1 and CREB5; KDF1 and AKT2; CREB5 and AKT2; CDK1, KDF1, and CREB5; CDK1, KDF1, and AKT2; CDK1, CREB5, and AKT2; KDF1, CREB5, and AKT2; or CDK1, KDF1, CREB5, and AKT2, and according to one embodiment of the present invention, CREB5 is a gene highly related to OLGIM stage and does not show a great correlation with mGC itself compared to other genes, but AKT2-isoform is a gene highly related to OLGIM stage and also shows a high correlation with mGC, so a combination of CDK1, KDF1, and AKT2-isoform can show the highest accuracy in predicting the possibility of developing metachronous gastric cancer, but is not limited to the combination of the above genes.

The composition of the present invention can obtain useful information for predicting the possibility of developing gastric cancer by measuring the increase in the expression of the above-mentioned gene, which has not been known until now and has been discovered for the first time by the present invention. Not only can it provide an approach for observing the occurrence of metachronous gastric cancer in the future for patients who have undergone endoscopic submucosal dissection for early gastric cancer, but it is also significant in that it is a quantitative method that can predict the prognosis and the possibility of developing gastric cancer in normal people, and thus it is possible to predict gastric cancer with a single measurement method using multiplex PCR with a single tissue sample.

In the present invention, “CDK1 (Cyclin Dependent Kinase 1)” is cyclin dependent kinase 1, also known as cell division cycle protein 2 homolog, which is a highly conserved protein that functions as a serine/threonine protein kinase and is a protein known to play a key role in cell cycle regulation. The CDK1 may comprise the amino acid sequence (SEQ ID NO: 1) of NCBI Reference Sequence: NP_001777.1 and may be encoded by the base sequence (SEQ ID NO: 2) of NCBI Reference Sequence: NM_001786.5, but is not limited thereto.

In one embodiment of the present invention, the ROC curve analysis result for determining the accuracy of predicting gastric cancer in a group of gastric cancer patients showed high accuracy (AUC 0.829), confirming that it is a biomarker that can be usefully used for predicting the likelihood of developing gastric cancer.

In the present invention, “KDF1 (Keratinocyte Differentiation Factor 1)” is a protein that plays a role in regulating epidermal formation during early development, and is known to act as an inhibitor of basal cell proliferation and a differentiation promoter of basal precursor cell progeny. The KDF1 may comprise the amino acid sequence (SEQ ID NO: 3) of NCBI Reference Sequence: NP_689578.2, and may be encoded by the base sequence (SEQ ID NO: 4) of NCBI Reference Sequence: NM_152365.3, but is not limited thereto.

In one embodiment of the present invention, the ROC curve analysis result for determining the accuracy of predicting gastric cancer in a group of gastric cancer patients showed very high accuracy (AUC 0.841), confirming that it is a biomarker that can be usefully used in predicting the likelihood of developing gastric cancer.

In the present invention, “CREB5 (CAMP responsive element binding protein 5)” is a gene belonging to the CRE-binding protein family and is known to act as a transcriptional activator of eukaryotic cells capable of regulating gene expression. The CREB5 may comprise the amino acid sequence (SEQ ID NO: 5) of NCBI Reference Sequence: NP_878901.2 and may be encoded by the base sequence (SEQ ID NO: 6) of NCBI Reference Sequence: NM_182898.4, but is not limited thereto.

In one embodiment of the present invention, it was confirmed that the expression of CREB5 increases as the stage increases in the OLGIM (Operative Link on Gastritis Assessment based on Intestinal Metaplasia) systems, which use intestinal metaplasia as a basis for evaluation, and in particular, it was confirmed that the expression amount of CREB5 significantly increases in stages 3 and 4, which are high-risk groups for gastric cancer development, thereby confirming that it is possible to predict the likelihood of gastric cancer development.

In the present invention, “AKT2 (AKT Serine/Threonine Kinase 2, RAC-beta serine/threonine-protein kinase)” is known to play a very important mediator role in the signal transduction pathway downstream of activated tyrosine kinase and PI3K, and cellular functions regulated by AKT are known to affect cellular functions such as cell proliferation, cell survival, cell size control, responsiveness to available nutrients, intermediate metabolic processes, angiogenesis, and tissue invasion. Specifically, AKT2 of the present invention may mean an AKT2 isoform, but is not limited thereto.

In the present invention, the AKT2 isoform may comprise an amino acid sequence of SEQ ID NO: 7 and may be encoded by a base sequence of SEQ ID NO: 8, but is not limited thereto.

In one embodiment of the present invention, as a result of measuring the expression of the AKT2 isoform, it was confirmed that the expression of the AKT2 isoform was significantly increased in a patient group with a high OLGIM stage compared to a patient group with a low OLGIM stage, and thus it was confirmed that it can be used to predict the likelihood of developing gastric cancer.

In the present invention, the “preparation for measuring the level of mRNA” means a preparation used in a method for measuring the level of mRNA transcribed from a gene in order to confirm whether the gene of the present invention contained in a sample is expressed.

In the present invention, the preparation for measuring the level of mRNA of the gene may be a primer or a probe that specifically binds to the gene or mRNA, but is not limited thereto.

In the present invention, a “primer” is a short single strand oligonucleotide that acts as a starting point for DNA synthesis. The primer specifically binds to a polynucleotide, which is a template, under suitable buffer and temperature conditions, and DNA is synthesized by DNA polymerase adding a nucleoside triphosphate having a base complementary to the template DNA to the primer and linking it. The primer generally consists of a sequence of 15 to 30 bases, and the temperature at which it binds to the template strand (melting temperature, Tm) varies depending on the base composition and length. The sequence of the primer does not need to be completely complementary to some of the base sequences of the template, and it is sufficient if it has a length and complementarity suitable for the purpose of measuring the amount of mRNA by amplifying a specific section of mRNA or cDNA through DNA synthesis. Therefore, in the present invention, a primer pair can be easily designed by referring to the base sequence of the cDNA or genomic DNA of the gene or its mRNA. The primers for the amplification reaction are composed of a set (pair) that complementarily bind to the template (or sense) and the opposite side (antisense) of both ends of a specific section of the mRNA to be amplified.

In the present invention, the “probe” means a fragment of a polynucleotide such as RNA or DNA, which has several to hundreds of base pairs long, and can specifically bind to mRNA, cDNA (complementary DNA), DNA, etc. of a specific gene, and the probe is labeled so that the presence or absence of the target mRNA or cDNA to which it binds, the expression level, etc. may be confirmed. The selection and hybridization conditions of the probe may be appropriately selected according to techniques known in the art. The probe may be used in a diagnostic method, etc. for detecting an allele (or allele). The diagnostic method comprises detection methods based on hybridization of nucleic acids, such as Southern blot, and may be provided in a form pre-bound to a substrate of a DNA chip in a method using a DNA chip.

In the present invention, the primer or probe may be chemically synthesized using a phosphoramidite solid support synthesis method or other widely known methods. In addition, the primer or probe may be variously modified according to methods known in the art within a range that does not interfere with hybridization with a polynucleotide that is a target to be detected. Examples of such modifications include methylation, capping, substitution with one or more homologs of a natural nucleotide, modification between nucleotides, for example, uncharged linkers (e.g., methyl phosphonate, phosphotriester, phosphoramidate, carbamate, etc.) or charged linkers (e.g., phosphorothioate, phosphorodithioate, etc.), and binding of labeling materials using fluorescent or enzymes.

In the present invention, the primer or probe is not limited to a specific sequence as long as it may detect the gene or its mRNA.

In the present invention, the preparation for measuring the level of the protein may be an antibody or an aptamer that is specific to the protein, but is not limited thereto.

In the present invention, the “aptamer” means a single-stranded nucleic acid (DNA, RNA, or modified nucleic acid) that has a stable tertiary structure and may bind to a target molecule with high affinity and specificity, and an aptamer for various desired target substances (proteins, sugars, dyes, DNA, metal ions, cells, etc.) may be developed by the method of Systematic Evolution of Ligands of Exponential enrichment (SELEX).

In the present invention, the “antibody” means a protein molecule that is directed to and specifically binds to an antigenic site. The antibody may be produced by methods commonly practiced in the art, such as fusion methods, recombinant DNA methods, or phage antibody library methods. In some embodiments, the antibody or antibody fragments may be derived from different organisms, comprising humans, mice, rats, hamsters, rabbits, or camels, etc. and may be, for example, monoclonal or polyclonal antibodies, immunologically active fragments, antibody heavy chains, humanized antibodies, antibody light chains, genetically engineered single-chain Fv molecules, or chimeric antibodies, etc. In the present invention, the antibody is not limited to a specific type of antibody as long as it may detect the protein of the present invention.

In the present invention, “gastric cancer” is a general term for cancer occurring in the stomach. Gastric adenocarcinoma, which accounts for the majority of gastric cancer, occurs in the glandular cells of the gastric mucosa and may be divided into several types according to the shape observed under a microscope. In addition, lymphoma occurring in lymphoid tissue, stromal tumor occurring in the nerve and muscle tissue of the stomach, sarcoma (malignant tumor derived from non-epithelial tissue), and neuroendocrine cancer that secretes hormones may be comprised in gastric cancer.

In the present invention, the gastric cancer may comprise both primary or metachronous gastric cancer, and accordingly, not only the occurrence of gastric cancer may be diagnosed and predicted even in normal people with a family history or risk factors, but metachronous gastric cancer may also be predicted.

In the present invention, “Metachronous Gastric Cancer (MGC)” refers to gastric cancer discovered 12 months after the initial diagnosis of gastric cancer, and more specifically, may refer to gastric cancer newly occurring in a site other than the original treatment site during follow-up observation after treatment with endoscopic submucosal dissection for early gastric cancer, but is not limited thereto, and is a concept that comprises any gastric cancer newly occurring after 12 months from the initial diagnosis of gastric cancer.

In addition, the present invention provides a composition for predicting the prognosis of early gastric cancer patients treated by endoscopic submucosal dissection, comprising a preparation for measuring the level of mRNA of any one or more genes selected from the group consisting of CDK1, KDF1, CREB5, and AKT2, or the level of proteins expressed therefrom.

In the present invention, “Endoscopic submucosal dissection” refers to a procedure for removing gastric polyps, adenomas, and early gastric cancer without lymph node metastasis using an endoscopic device.

In one embodiment of the present invention, the expression of CDK1, KDF1, CREB5, or AKT2 was confirmed in early gastric cancer patients treated by endoscopic submucosal dissection to predict the likelihood of developing metachronous gastric cancer, and through this, it was confirmed that the prognosis of early gastric cancer patients after endoscopic submucosal dissection treatment may be predicted.

In addition, the present invention provides a use of a composition comprising a preparation for measuring the level of mRNA of any one or more genes selected from the group consisting of CREB5 and AKT2, or the level of proteins expressed therefrom for predicting the likelihood of developing gastric cancer.

In addition, the present invention provides a use of a preparation for measuring the level of mRNA of any one or more genes selected from the group consisting of CREB5 and AKT2, or the level of proteins expressed therefrom for manufacturing a preparation for predicting the likelihood of developing gastric cancer.

In addition, the present invention provides a use of a composition comprising a preparation for measuring the level of mRNA of any one or more genes selected from the group consisting of CDK1, KDF1, CREB5, and AKT2, or the level of proteins expressed therefrom for predicting the likelihood of developing metachronous gastric cancer.

In addition, the present invention provides a use of a preparation for measuring the level of mRNA of any one or more genes selected from the group consisting of CDK1, KDF1, CREB5, and AKT2, or the level of proteins expressed therefrom for manufacturing a preparation for predicting the likelihood of developing metachronous gastric cancer.

In addition, the present invention provides a use of a composition comprising a preparation for measuring the level of mRNA of any one or more genes selected from the group consisting of CDK1, KDF1, CREB5, and AKT2, or the level of proteins expressed therefrom for predicting the prognosis of early gastric cancer patients treated by endoscopic submucosal dissection.

In addition, the present invention provides a use of a preparation for measuring the level of mRNA of any one or more genes selected from the group consisting of CDK1, KDF1, CREB5, and AKT2, or the level of proteins expressed therefrom for manufacturing a preparation for predicting the prognosis of early gastric cancer patients treated by endoscopic submucosal dissection.

In addition, the present invention provides a kit for predicting the likelihood of developing gastric cancer or metachronous gastric cancer, comprising the composition.

In the present invention, the “kit” means a tool that can predict the likelihood of developing gastric cancer or metachronous gastric cancer by comprising a preparation for measuring mRNA or proteins of one or more genes selected from the group consisting of CDK1, KDF1, CREB5, and AKT2. In the present invention, the kit may be one or more selected from the group consisting of a reverse transcription polymerase chain reaction (RT-PCR) kit, a real-time polymerase chain reaction (qRT-PCR) kit, a DNA chip kit, an Enzyme-linked immunosorbent assay (ELISA) kit, and a protein chip kit, but is not limited thereto.

In addition to the preparation for measuring the mRNA or protein, the kit of the present invention may comprise other components, compositions, solutions, devices, etc. that are usually required for the detection method thereof. At this time, the substance for detecting the expression level of the mRNA or protein may be applied at least once without limitation in the number of times, and there is no limitation on the order in which each substance is applied, and the application of each material may be performed simultaneously or randomly.

Specifically, the kit for measuring the mRNA expression level of the gene of the present invention may be a kit comprising essential elements necessary for performing RT-PCR. The RT-PCR kit may comprise, in addition to each primer pair specific for the gene, a test tube or other appropriate container, a reaction buffer (with various pH and magnesium concentrations), deoxynucleotides (dNTPs), enzymes such as Taq polymerase and reverse transcriptase, DNase, RNAse inhibitor, DEPC-water, sterile water, etc. In addition, it may comprise a primer pair specific for the gene used as a quantitative control.

In addition, the kit of the present invention may comprise essential elements necessary for performing a DNA chip analysis method. The DNA chip analysis kit may comprise a substrate to which a cDNA corresponding to a gene or a fragment thereof is attached as a probe, and reagents, preparations, enzymes, etc. for producing a fluorescent label probe. In addition, the substrate may comprise a cDNA corresponding to a quantitative control gene or a fragment thereof.

In addition, the kit of the present invention may be a protein chip analysis kit for measuring the level of a protein encoded from the gene, and the kit is not particularly limited thereto, but may comprise a material, an appropriate buffer solution, a secondary antibody labeled with a chromogenic enzyme or fluorescent substance, a chromogenic substrate, etc. for immunological detection of an antibody. The material is not particularly limited thereto, but a nitrocellulose membrane, a 96-well plate synthesized with polyvinyl resin, a 96-well plate synthesized with polystyrene resin, and a glass slide glass may be used, and the chromogenic enzyme is not particularly limited thereto, but peroxidase and alkaline phosphatase may be used, and the fluorescent substance is not particularly limited thereto, but may be FITC, RITC, etc., and the chromogenic substrate solution is not particularly limited thereto, but may be ABTS (2,2′-azino-bis(3-ethylbenzothiazoline-6-sulfonic acid)) or OPD (o-phenylenediamine), TMB (tetramethyl benzidine).

In the present invention, the kit may comprise a container; instructions; and a detection preparation of the mRNA or protein. The container may serve to package the detection preparation, and may also serve to store and fix the detection preparation. The material of the container may take the form of, for example, a bottle, a tub, a sachet, an envelope, a tube, an ampoule, and the like, which may be formed partly or wholly from plastics, glass, paper, foil, wax, and the like. The container may be initially equipped with a completely or partially detachable stopper, which is part of the container or which may be attached to the container by mechanical, adhesive, or other means. Also, the container may also be equipped with a stopper through which the contents may be accessed by a syringe needle. The kit may comprise an outer package, and the outer package may include instructions regarding the use of the components.

In addition, the present invention provides a method for providing information for predicting the likelihood of developing gastric cancer; or a method for predicting the likelihood of developing gastric cancer, comprising: (a) measuring the level of mRNA of any one or more genes selected from the group consisting of CREB5 and AKT2, or the level of proteins expressed therefrom in a biological sample isolated from a subject; and
(b) comparing the measured mRNA or protein level to the level measured in a biological sample isolated from a control group.

In the present invention, the gastric cancer may comprise metachronous gastric cancer, and the method may further comprise measuring the level of mRNA of any one or more genes selected from the group consisting of CDK1 and KDF1, or the level of proteins expressed therefrom in a biological sample isolated from the subject, and comparing the level to that measured in a sample isolated from a control group, but is not limited thereto.

In addition, the present invention provides a method for providing information for predicting the likelihood of developing metachronous gastric cancer; or a method for predicting the likelihood of developing metachronous gastric cancer, comprising: (a) measuring the level of mRNA of any one or more genes selected from the group consisting of CDK1, KDF1, CREB5, and AKT2, or the level of proteins expressed therefrom in a biological sample isolated from a subject; and
(b) comparing the measured mRNA or protein level to the level measured in a biological sample isolated from a control group.

In addition, the present invention provides a method for providing information for predicting the prognosis of early gastric cancer patients after treatment by endoscopic submucosal dissection; or a method for predicting the prognosis of early gastric cancer patients after treatment by endoscopic submucosal dissection, comprising: (a) measuring the level of mRNA of any one or more genes selected from the group consisting of CDK1, KDF1, CREB5, and AKT2, or the level of proteins expressed therefrom in a biological sample isolated from early gastric cancer patients; and
(b) comparing the measured mRNA or protein level to the level measured in a biological sample isolated from a control group.

In the present invention, the level of mRNA of the gene may be measured by one or more methods selected from the group consisting of reverse transcription polymerase chain reaction (RT-PCR), competitive reverse transcription polymerase chain reaction (competitive RT-PCR), real-time quantitative reverse transcription polymerase chain reaction (real-time quantitative RT-PCR), multiplex reverse transcription polymerase chain reaction (Multi-plex PCR), real-time polymerase chain reaction (qRT-PCR), RNase protection method, Northern blotting, DNA chip technology assay, methylated DNA binding domain sequencing (MBD-seq), and reduced representation bisulfite sequencing (RRBS), but is not limited thereto.

In the present invention, the composition may analyze the expression levels of four genes at once through multiplex reverse transcriptase polymerase reaction (multi-plex PCR), but is not limited thereto.

In the present invention, the level of the protein may be measured by one or more methods selected from the group consisting of western blot, enzyme-linked immunosorbent assay (ELISA), radioimmunoassay (RIA), radioimmunodiffusion, ouchterlony immunodiffusion, rocket immunoelectrophoresis, immunohistochemistry (IHC), immunoprecipitation assay, complement fixation assay, fluorescence-activated cell sorting (FACS), and protein chip, but is not limited thereto.

In the present invention, the measurement of the mRNA level or protein level of the gene may be measured using a single biological sample, but is not limited thereto.

In the present invention, the biological sample means any sample that the presence or absence of expression or the expression level of mRNA or protein of CDK1, KDF1, CREB5, or AKT2 in the body may be confirmed, and for example, the biological sample may be one or more selected from the group consisting of tissue, cells, whole blood, serum, plasma, saliva, sputum, cerebrospinal fluid, urine, and feces isolated from the subject, but is not limited thereto. Specifically, the tissue may be gastric mucosa (GM) tissue or intestinal metaplasia (IM) tissue, but is not limited thereto.

In the present invention, the method for providing information for predicting the likelihood of developing gastric cancer or metachronous gastric cancer may further comprise (c) determining that the risk of developing gastric cancer or metachronous gastric cancer is high when the level of the mRNA or protein measured in the biological sample isolated from the subject is higher than the level measured in a biological sample isolated from the control group, but is not limited thereto.

In the present invention, the method for providing information for predicting the prognosis of early gastric cancer patients after treatment by endoscopic submucosal dissection may further comprise (c) predicting that the prognosis after endoscopic submucosal dissection treatment will be poor when the level of the mRNA or protein measured in the biological sample isolated from early gastric cancer patients is higher than the level measured in a biological sample isolated from the control group, but is not limited thereto. In this case, the biological sample may be gastric mucosal tissue around a tumor of an early gastric cancer patient, and may be separated before, after, or regardless of the elapsed time of endoscopic submucosal dissection treatment, but is not limited thereto.

In addition, the present invention provides a method for predicting the likelihood of developing gastric cancer, comprising: (a) manufacturing a preparation for measuring the level of mRNA of any one or more genes selected from the group consisting of CREB5 and AKT2, or the level of proteins expressed therefrom;
(b) measuring the level of mRNA of any one or more genes selected from the group consisting of CREB5 and AKT2, or the level of proteins expressed therefrom in a biological sample isolated from a subject using the preparation; and
(c) comparing the measured mRNA or protein level to the level measured in a biological sample isolated from a control group.

In addition, the present invention provides a method for predicting the likelihood of developing metachronous gastric cancer, comprising: (a) manufacturing a preparation for measuring the level of mRNA of any one or more genes selected from the group consisting of CDK1, KDF1, CREB5, and AKT2, or the level of proteins expressed therefrom;
(b) measuring the level of mRNA of any one or more genes selected from the group consisting of CDK1, KDF1, CREB5, and AKT2, or the level of proteins expressed therefrom in a biological sample isolated from a subject using the preparation; and
(c) comparing the measured mRNA or protein level to the level measured in a biological sample isolated from a control group.

In addition, the present invention provides a method for predicting the prognosis of early gastric cancer patients after treatment by endoscopic submucosal dissection, comprising: (a) manufacturing a preparation for measuring the level of mRNA of any one or more genes selected from the group consisting of CDK1, KDF1, CREB5, and AKT2, or the level of proteins expressed therefrom;
(b) measuring the level of mRNA of any one or more genes selected from the group consisting of CDK1, KDF1, CREB5, and AKT2, or the level of proteins expressed therefrom in a biological sample isolated from early gastric cancer patients using the preparation; and
(c) comparing the measured mRNA or protein level to the level measured in a biological sample isolated from a control group.

In the present invention, the preparation for measuring the level of mRNA of the gene or protein expressed therefrom may be a primer or a probe that specifically binds to the gene or mRNA; or an antibody or an aptamer that is specific to the protein, but is not limited thereto.

In the present invention, “prediction” may mean confirming the likelihood or risk of developing gastric cancer or metachronous gastric cancer for the purpose of the present invention, or confirming the prognosis after endoscopic submucosal dissection treatment of an early gastric cancer patient.

In the present invention, “subject” means a subject whose development of gastric cancer or recurrence of gastric cancer is to be predicted, or a subject diagnosed with early gastric cancer and for whom the likelihood or prognosis of developing metachronous gastric cancer is to be predicted before or after endoscopic submucosal dissection treatment. In the present invention, the subject or individual may comprise, without limitation, any animal that can develop gastric cancer, comprising humans, dogs, horses, cows, mice, goats, rabbits, chickens, ducks, geese, etc.

In the present invention, “control group” comprises an individual not diagnosed with gastric cancer, and an individual diagnosed with early gastric cancer and not developing metachronous gastric cancer after endoscopic submucosal dissection treatment. According to the present invention, the likelihood of developing gastric cancer in a subject may be predicted by comparing a sample isolated from an individual not diagnosed with gastric cancer with a sample isolated from a subject whose likelihood of developing gastric cancer is to be predicted, and the likelihood of developing metachronous gastric cancer in a subject may be predicted by comparing a sample isolated from an individual who has not developed metachronous gastric cancer after endoscopic submucosal dissection treatment with a sample isolated from a subject whose possibility of developing metachronous gastric cancer is to be predicted.

The method for providing information for predicting the likelihood of developing gastric cancer of the present invention may be measured with a single sample by collecting a sample once instead of the existing method of collecting samples from an individual multiple times to measure the expression levels of the four types of genes, and the diagnosis and prediction of gastric cancer may be made with only one measurement by measuring the gene expression levels using multiplex reverse transcriptase polymerase reaction.

In addition, the method may be usefully used not only to predict metachronous gastric cancer, but also to predict the risk of developing gastric cancer in normal people.

In the present invention, when the term “comprising” is used, it means that other components can be included rather than excluding other components unless otherwise specifically stated. The terms “step of ~” used throughout the present invention do not mean “step for ~.”

Hereinafter, the present invention will be described in more detail through the following examples. However, these examples are intended to exemplify the present invention and the scope of the present invention is not limited to these examples.

### EXAMPLE

### Example 1. Selection of Candidate Genes for Diagnosis and Development Prediction of Gastric Cancer

In order to find biomarkers for predicting gastric cancer development through genome analysis of gastric mucosal tissue in early gastric cancer patients treated by endoscopic submucosal dissection, normal gastric mucosal tissue was obtained at the time of diagnosis from early gastric cancer patients treated by endoscopic submucosal dissection and stored in an ultra-low temperature freezer. Among these subjects, 23 subjects who developed metachronous gastric cancer during follow-up and 23 subjects who did not develop new gastric cancer or gastric adenomas during a follow-up period of more than 3 years were selected.

RNA sequencing was performed on the normal gastric mucosal tissue stored in an ultra-low temperature freezer at the time of diagnosis from the selected subjects to identify differentially expressed genes (DEG).

In addition, a correlation analysis was performed according to the level of gene expression and the number of metachronous gastric cancers, the number of adenomas, the degree of atrophic gastritis and intestinal metaplasia, and candidate genes were selected by performing self-organizing map (SOM) analysis and isoform analysis.

Meanwhile, whole exome sequencing (WES) was performed on tumor tissues using the formalin-fixed paraffin-embedded endoscopic submucosal dissection tissue of the subject. A real-time polymerase chain reaction (qRT-PCR) was performed on various gastric cancer cell lines and normal gastric mucosa cell lines for candidate genes. When compared with the normal gastric mucosa cell line, genes showing significant differences in gastric cancer cell lines were selected. The gastric cancer cell lines were treated with siRNA for the selected genes, and the difference in cell viability was evaluated.

In order to evaluate the validation, a validation evaluation set was constructed independently of the above 46 subjects, consisting of 50 subjects who developed metachronous gastric cancer after endoscopic submucosal dissection and 100 subjects who did not develop metachronous gastric cancer. Real-time qPCR, Western blot, and immunohistochemistry were performed for the candidate genes to compare them.

RNA sequencing was performed on normal gastric mucosal tissues stored in an ultra-low temperature freezer after a biopsy was performed on the selected subjects at the time of diagnosis, and differentially expressed genes (DEG) were identified using DESeq2. During the analysis process, the batch effect by Helicobacter pylori was confirmed, and batch correction was performed.

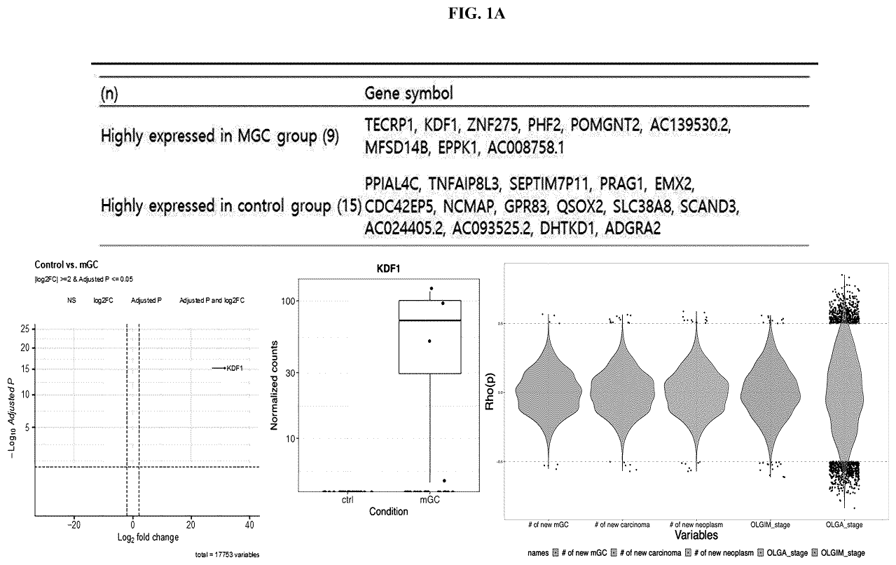

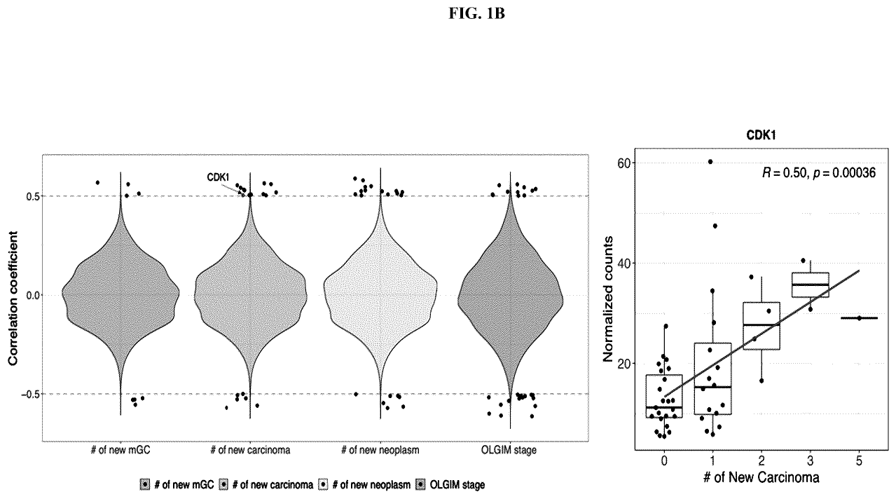

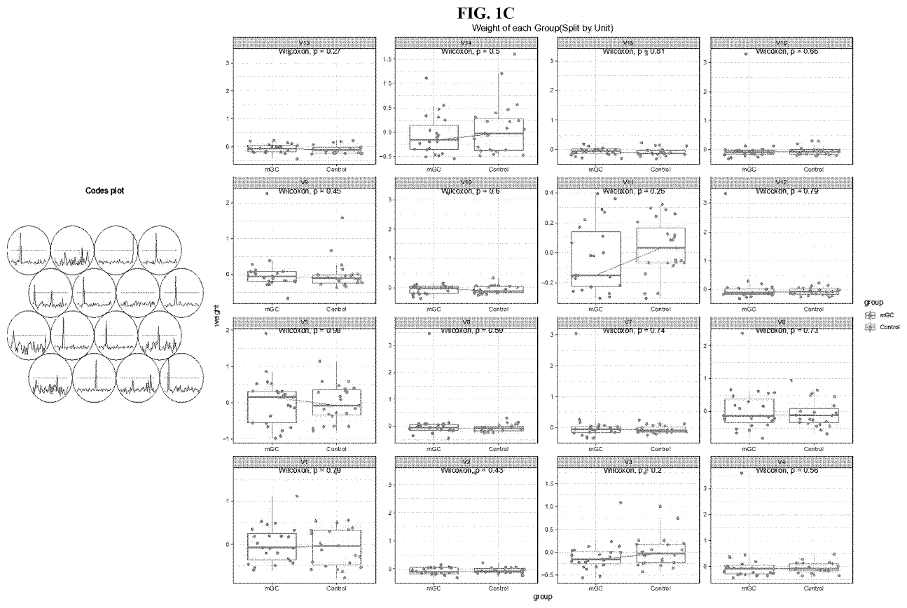

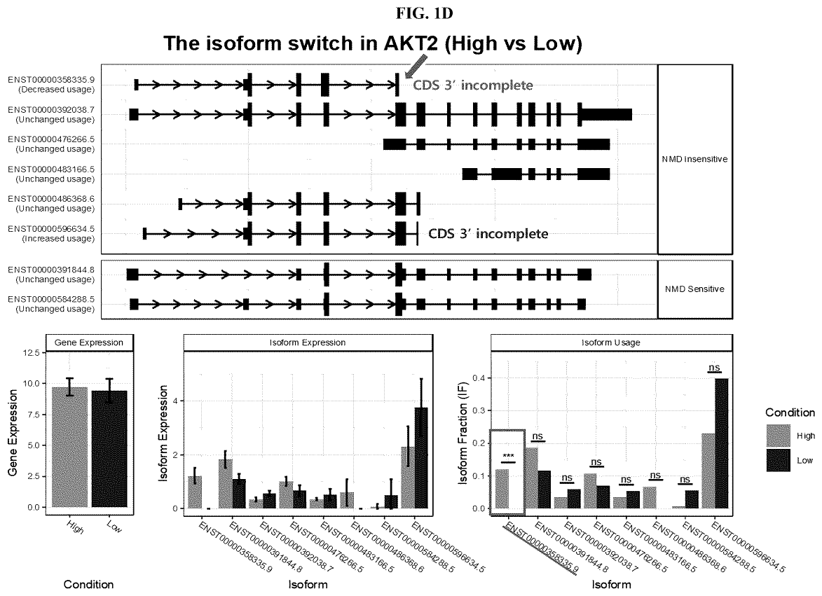

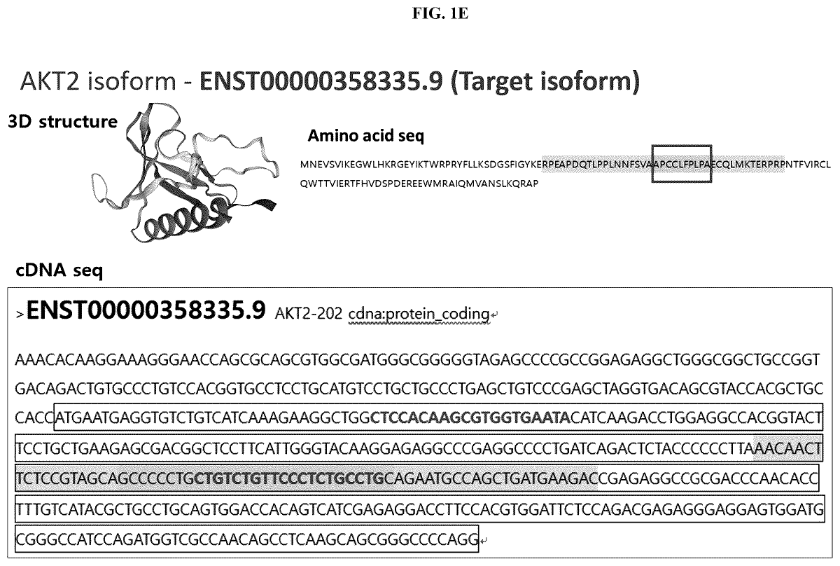

As a result, a total of 24 DEGs were identified, and 9 DEGs were significantly increased in the mGC group (subjects who developed metachronous gastric cancer after endoscopic submucosal dissection treatment) and 15 DEGs were significantly increased in the control group (subjects who did not develop metachronous gastric cancer after endoscopic submucosal dissection treatment) (FIG. 1A).

In addition, the clinical information of the target patient group was reviewed, and a correlation analysis between the number of metachronous gastric cancers, the number of adenomas, neoplasms (gastric cancer+adenomas), the degree of atrophic gastritis (OLGA stage), and the degree of intestinal metaplasia (OLGMI stage) and the expression level of each gene confirmed using DESeq2 was performed, and self-organizing map (SOM) and alternative splicing analysis were performed.

As a result, although no significant difference was observed according to mGC in SOM analysis, a common significant correlation was confirmed in unit V7 including CREB5 when analyzed with OLGIM stage and MLH-1 IHC (FIG. 1C).

In the isoform analysis confirmed by alternative splicing, there was no significant isoform difference between the mGC group and the control group, but when the OLGA stage and OLGIM stage were analyzed, significant differences were observed in the isoform of AKT2 (FIG. 1D) and the isoform of KDM5C. At this time, the cDNA sequence (SEQ ID NO: 7) and amino acid sequence (SEQ ID NO: 8) of the AKT2 isoform targeted for which there was a significant difference in expression depending on the OLGIM stage are shown in FIG. 1E.

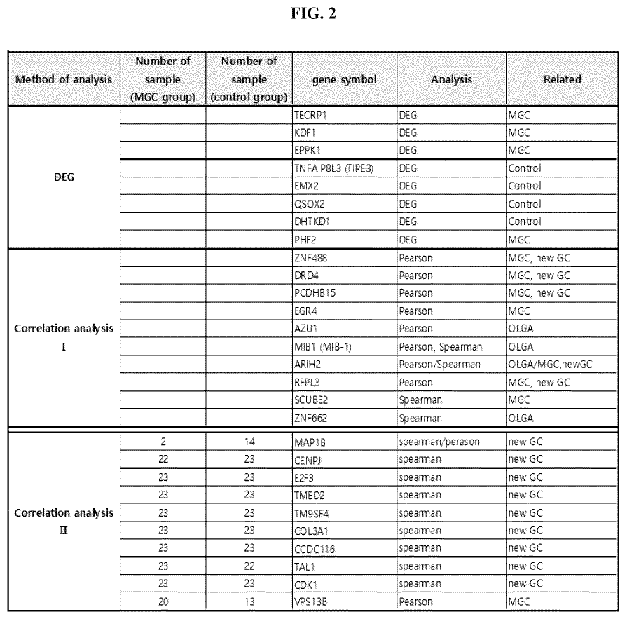

Based on the results of bulk RNA sequencing analysis performed on normal gastric mucosal tissue surrounding the tumor, additional correlation analysis, self-organizing map, and isoform analysis performed based on the results of the analysis, and the functions and reporting frequencies of the genes identified in previous studies, CDK1, KDF1, ERCC6L, KLK8, MCM2, POLR2L, PTN, CREB5, and AKT2-isoform were selected as several candidate genes (FIG. 2).

### Example 2. Target Gene Selection and Protein Expression Confirmation Through Confirmation of Candidate Gene Expression Level in Gastric Cancer Cell Lines

2-1. Confirmation of Gene Expression Through qRT-PCR

For candidate genes selected by RNA sequencing performed with normal gastric mucosal tissue around the tumor and candidate genes selected by WES of the tumor, the degree of gene expression was compared by performing real-time polymerase chain reaction (qRT-PCR) for various gastric cancer cell lines (intestinal type: SNU216, MKN1, AGS, diffuse type: SNU1, SNU5, SNU16, SNU488, SNU601, SNU620, SNU638, MKN45) and normal gastric mucosal cell lines (HFE145).

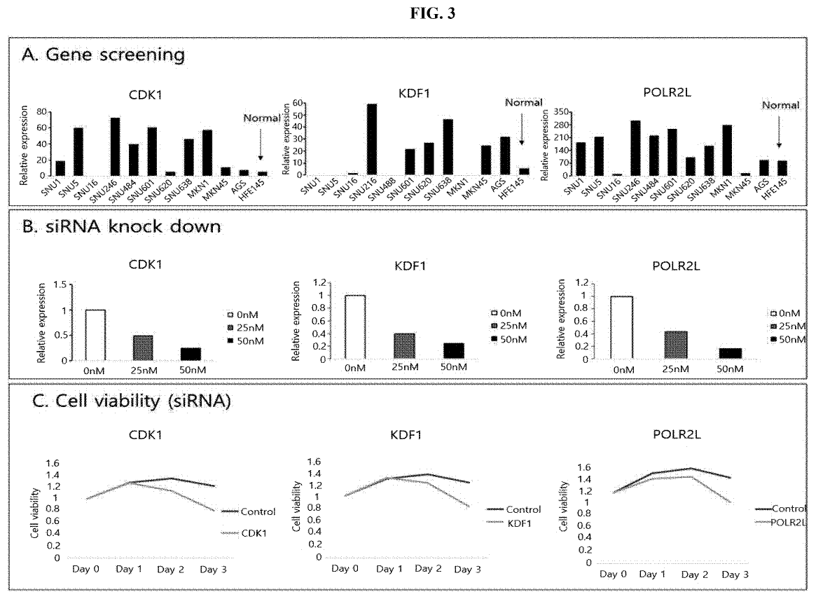

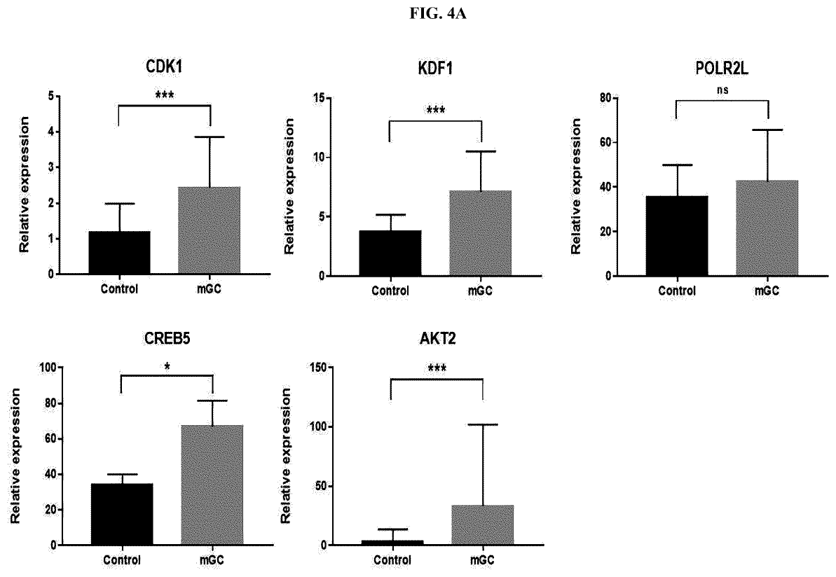

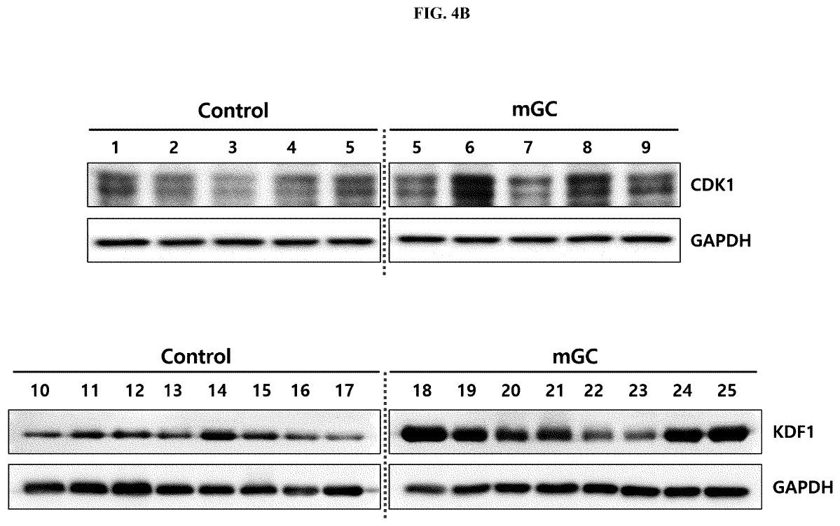

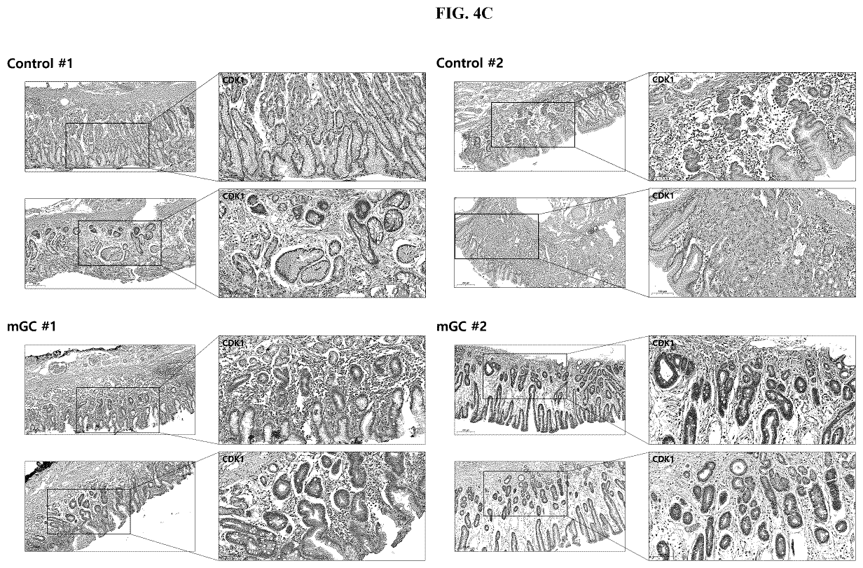

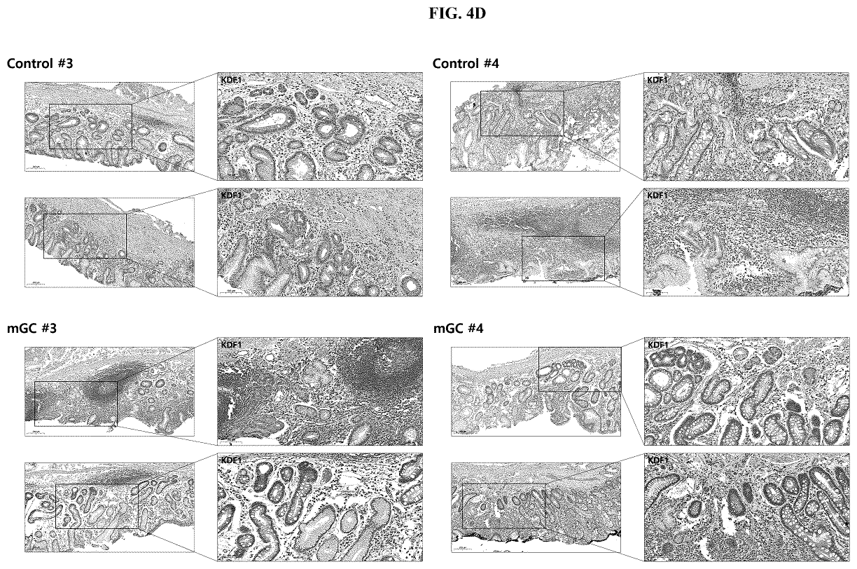

The gastric cancer cell lines were treated with siRNA (0 nM, 25 nM, 50 nM) for the selected genes to confirm the inhibition of gene expression, and the cell viability inhibition in the siRNA-treated gastric cancer cell lines was evaluated and compared, and three target genes (CDK1, KDF1, POLR2L) showing high expression in the mGC group were selected, including CDK1 (FIG. 1B), which was confirmed to have higher expression in the mGC group than in the control group by correlation analysis (FIG. 1A, FIG. 1B, and FIG. 3). Then, among a total of five genes (CREB5, AKT2-isoform, CDK1, KDF1, and POLR2L) in FIGS. 1A to 1E and FIG. 3, CDK1, KDF1, CREB5, and AKT2-isoform were selected as the four final target genes, excluding POLR2L, which did not have a significant difference in expression between control and mGC (FIG. 4A).

### 2-2. Confirmation of CDK1 and KDF1 Protein Expression Through Western Blot

Among the evaluation sets used in FIG. 4A, proteins were isolated from the tissues of some patients, and the protein expression levels of CDK1 and KDF1, which showed high expression in the mGC group, were confirmed through western blot.

5 subjects who developed metachronous gastric cancer and 5 subjects who did not develop metachronous gastric cancer after endoscopic submucosal dissection treatment were randomly selected, and the protein expression of CDK1 was confirmed. As a result, it was confirmed that the expression of CDK1 was higher in the tissues of the subjects who developed metachronous gastric cancer than in the tissues of the subjects who did not develop metachronous gastric cancer (top of FIG. 4B).

In addition, as a result of randomly selecting 8 patients in each group and confirming the protein expression of KDF1, as with CDK1, it was confirmed that the expression of KDF1 was higher in the tissues of the subjects who developed metachronous gastric cancer than in the tissues of the subjects who did not develop metachronous gastric cancer (bottom of FIG. 4B).

These results were consistent with the results of previously confirming mRNA expression levels through qPCR, indicating that both CDK1 and KDF1 were expressed more highly in subjects who developed metachronous gastric cancer.

### 2-3. Confirmation of CDK1 and KDF1 Protein Expression Through Immunohistochemistry (IHC)

The expression of the previously selected candidate genes CDK1 and KDF1 on the pathology slides of some patients among the evaluation sets used in FIG. 4A was confirmed using immunohistochemistry.

After endoscopic submucosal dissection treatment, two subjects who developed metachronous gastric cancer and two subjects who did not develop metachronous gastric cancer were randomly selected and the protein expression of CDK1 (FIG. 4C) and KDF1 (FIG. 4D) was confirmed. As a result, both candidate genes were stained more strongly in the tissues of the subjects who developed metachronous gastric cancer than in the tissues of the subjects who did not develop metachronous gastric cancer (FIGS. 4C and 4D). This means that the proteins of CDK1 and KDF1 are more abundant in the tissues of the subjects who developed metachronous gastric cancer, which is the same result as the qPCR results of Example 2-1 and the Western blot results of Example 2-2.

### Example 3. Evaluation of Target Gene Validation

In order to evaluate a validation of the target genes selected in Examples 1 and 2 above, a validation evaluation set independent of the 46 patient group set for candidate gene selection was established. The set consists of 50 subjects who developed metachronous gastric cancer after endoscopic submucosal dissection treatment and 100 subjects who did not develop metachronous gastric cancer, and were selected by matching age, sex, and H. pylori infection status.

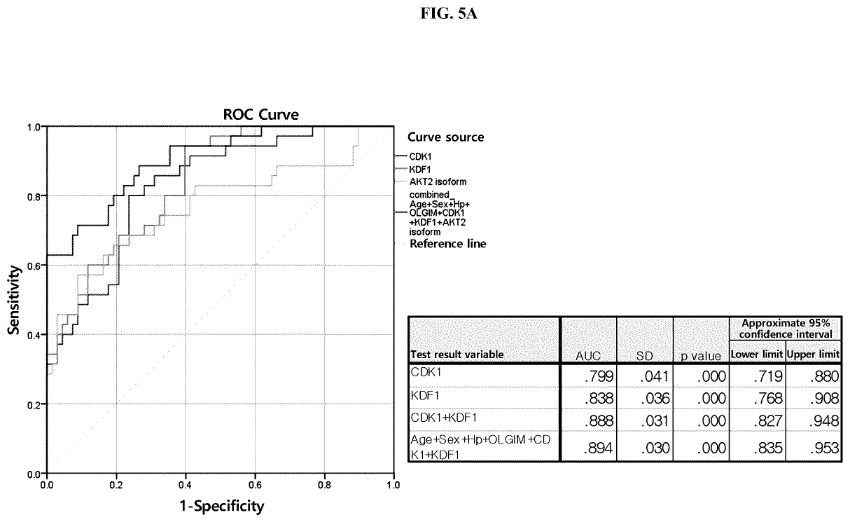

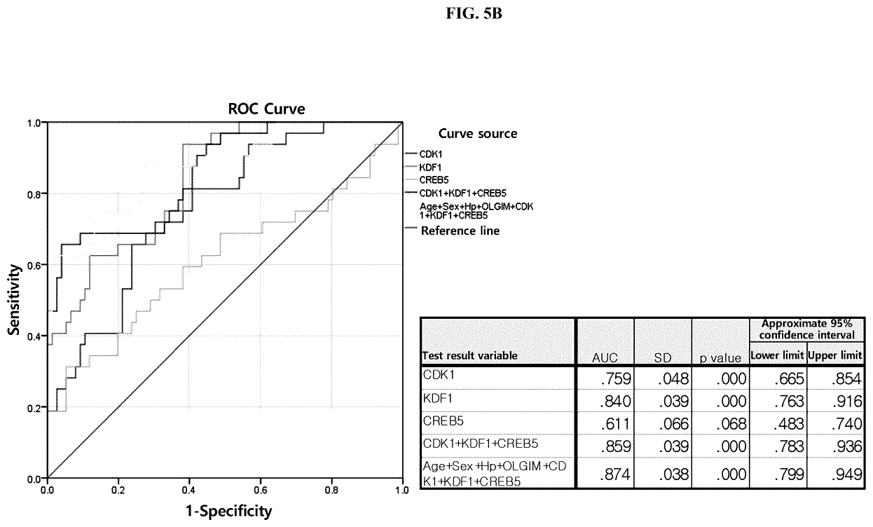

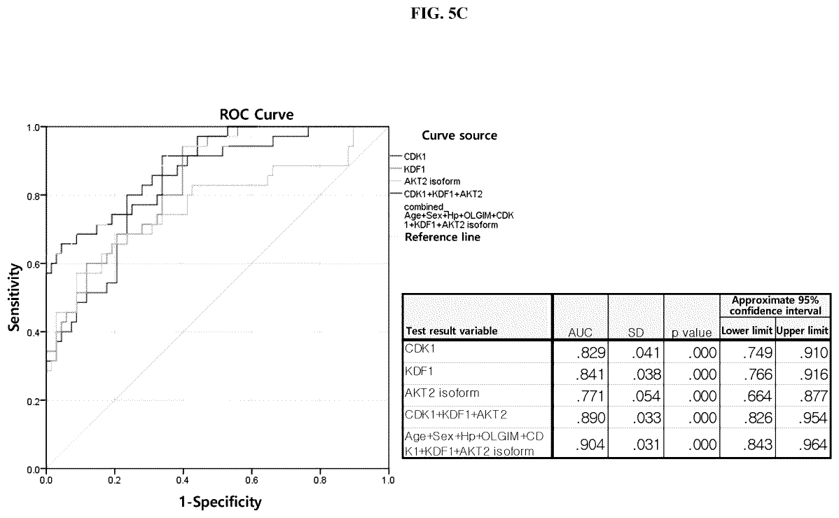

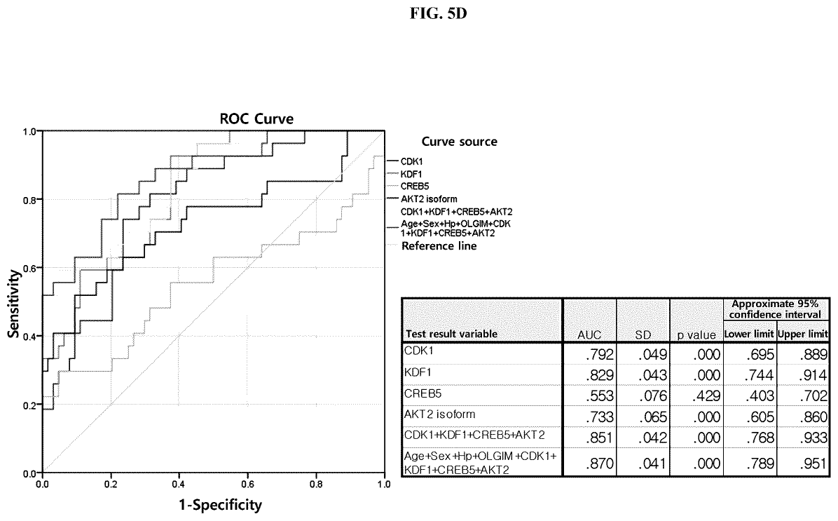

In order to determine the accuracy of predicting gastric cancer in the patient group using the four genes selected in Example 2 above (CDK1, KDF1, CREB5, and AKT2-isoform), a ROC curve was drawn (FIGS. 5A to 5D), as a result, the gene combinations that showed the highest accuracy are shown in Table 1 and FIG. 5C.

TABLE 1

| Gene | AUC | SD | 95% CI | P value |
|---|---|---|---|---|
| CDK1 | 0.829 | 0.041 | 0.749~0.910 | <0.000 |
| KDF1 | 0.841 | 0.038 | 0.766~0.916 | <0.000 |
| AKT2-isoform | 0.771 | 0.054 | 0.664~0.877 | <0.000 |
| CDK1 + KDF1 + AKT2-isoform | 0.890 | 0.033 | 0.826~0.954 | <0.000 |
| Age + Sex + Hp + OLGIM + | 0.904 | 0.031 | 0.843~0.964 | <0.000 |
| CDK1 + KDF1 + AKT2-isoform | | | | |

As shown in the results in Table 1 above, as a result of the CDK1 ROC curve analysis, the accuracy was AUC 0.829, the AUC of the KDF1 ROC curve analysis result was 0.841, and the AUC of the AKT2-isoform ROC curve was 0.771, confirming that the accuracy of each gene in predicting gastric cancer was high.

In addition, the expression levels of the combination of the CDK1, KDF1, and AKT2 isoforms were measured, and the ROC curve was analyzed. As a result, the accuracy was significantly increased with an AUC of 0.890, confirming that the accuracy of predicting gastric cancer increased when the four genes were used in combination compared to when they were used individually.

Next, RNA was extracted and qPCR was performed to confirm whether CREB5 and AKT2, which showed expression differences according to the OLGIM (Operative Link on Gastritis Assessment based on Intestinal Metaplasia) stage in bulk RNA sequencing data, showed the same results in the 150-person evaluation set. In the case of the OLGIM stage, the degree and extent of atrophy are evaluated and divided into five stages (stages 0-4), and it has been reported in several studies that it is used as an index to predict the likelihood of gastric cancer along with the OLGA stage.

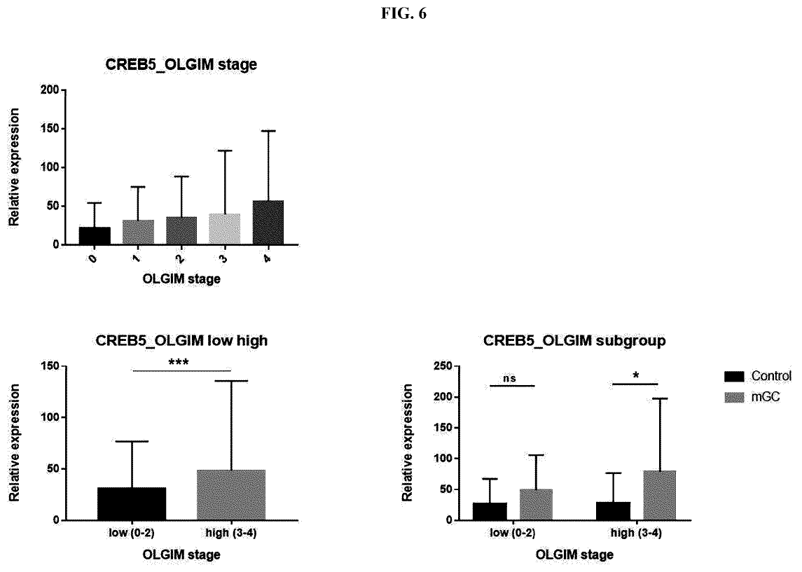

First, in the case of CREB5, it was confirmed that the expression of CREB5 appeared strongly as the OLGIM stage increased (FIG. 6). That is, in the OLGIM system that uses intestinal metaplasia as the basis for evaluation, it was confirmed that the expression of CREB5 was significantly increased in stages 3 and 4, which are high-risk groups for development of gastric cancer, confirming that gastric cancer prediction is possible.

In addition, as a result of analyzing alternative splicing, the total expressed AKT2 RNA amount was not significantly related to the OLGIM stage, but it was confirmed that the expression of specific isoforms greatly differed depending on the OLGIM stage (FIG. 1D). Thus, the evaluation set samples were divided into patient groups with high and low OLGIM stages and experiments were conducted.

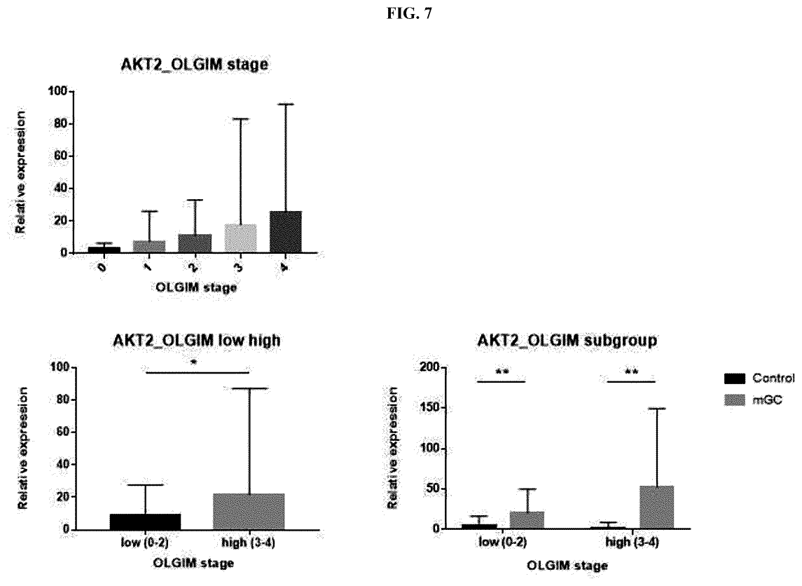

The expression of specific AKT2 isoforms confirmed previously in each group was confirmed through qPCR. As a result, it was confirmed that the expression of specific AKT2 isoforms was significantly higher in the patient group with high OLGIM stage compared to the patient group with low OLGIM stage (FIG. 7).

From the above results, the possibility of utilizing CDK1, KDF1, CREB5, and AKT2 isoforms as predictive factors for the likelihood of developing gastric cancer was confirmed, and it is possible to utilize them as predictive markers for the development of metachronous or primary gastric cancer depending on the increased expression of the above genes. Furthermore, the prediction of the likelihood of developing gastric cancer according to the present invention may be confirmed with a single tissue sample, and it has a technical advantage in that it is highly utilized because it is possible to predict the likelihood of developing gastric cancer in a single measurement method using multi-plex PCR. In addition, since it comprises a method for quantifying basal gastritis in a quantitative method that can predict the development of gastric cancer not only in patients with metachronous gastric cancer but also in normal people, it simultaneously provides a method for predicting the risk of development of gastric cancer in normal people with a family history of gastric cancer or risk factors. In addition, by predicting the development of metachronous gastric cancer in patients with early gastric cancer treated by endoscopic submucosal dissection, the prognosis of patients after endoscopic submucosal dissection treatment may be predicted.

### INDUSTRIAL APPLICABILITY

Any one or more genes selected from the group consisting of CDK1, KDF1, CREB5, and AKT2 according to the present invention are expected to be useful for predicting the likelihood of developing gastric cancer or metachronous gastric cancer, or predicting the prognosis in early gastric cancer patients treated by endoscopic submucosal dissection, and thus have industrial applicability.

1.-19. (canceled)

20. A method for predicting the likelihood of developing gastric cancer, comprising:
(a) manufacturing a preparation for measuring the level of mRNA of any one or more genes selected from the group consisting of CREB5 and AKT2, or the level of proteins expressed therefrom;
(b) measuring the level of mRNA of any one or more genes selected from the group consisting of CREB5 and AKT2, or the level of proteins expressed therefrom in a biological sample isolated from a subject using the preparation; and
(c) comparing the measured mRNA or protein level to the level measured in a biological sample isolated from a control group.

21. The method for predicting the likelihood of developing gastric cancer of claim 20, wherein the gastric cancer comprises metachronous gastric cancer.

22. The method for predicting the likelihood of developing gastric cancer of claim 21, wherein the method further comprises measuring the level of mRNA of any one or more genes selected from the group consisting of CDK1 and KDF1, or the level of proteins expressed therefrom in a biological sample isolated from the subject, and comparing the level to that measured in a sample isolated from a control group.

23. The method for predicting the likelihood of developing gastric cancer of claim 20, wherein the level of mRNA of the gene is measured by one or more methods selected from the group consisting of reverse transcription polymerase chain reaction (RT-PCR), competitive reverse transcription polymerase chain reaction (competitive RT-PCR), real-time quantitative reverse transcription polymerase chain reaction (real-time quantitative RT-PCR), multiplex reverse transcription polymerase chain reaction (Multi-plex PCR), real-time polymerase chain reaction (qRT-PCR), RNase protection method, Northern blotting, DNA chip technology assay, methylated DNA binding domain sequencing (MBD-seq), and reduced representation bisulfite sequencing (RRBS).

24. The method for predicting the likelihood of developing gastric cancer of claim 20, wherein the level of the protein is measured by one or more methods selected from the group consisting of western blot, enzyme-linked immunosorbent assay (ELISA), radioimmunoassay (RIA), radioimmunodiffusion, ouchterlony immunodiffusion, rocket immunoelectrophoresis, immunohistochemistry (IHC), immunoprecipitation assay, complement fixation assay, fluorescence-activated cell sorting (FACS), and protein chip.

25. The method for predicting the likelihood of developing gastric cancer of claim 20, wherein the measurement of the mRNA level or protein level of the gene is measured using a single biological sample.

26. The method for predicting the likelihood of developing gastric cancer of claim 20, wherein the biological sample is one or more selected from the group consisting of tissue, cells, whole blood, serum, plasma, saliva, sputum, cerebrospinal fluid, urine, and feces isolated from the subject.

27. The method for predicting the likelihood of developing gastric cancer of claim 20, wherein the method further comprises:
(d) determining that the risk of developing gastric cancer is high when the level of the mRNA or protein measured in the biological sample isolated from the subject is higher than the level measured in a biological sample isolated from the control group.

28. The method for predicting the likelihood of developing gastric cancer of claim 20, wherein the preparation for measuring the mRNA level of the gene is a primer or a probe that specifically binds to the gene or mRNA.

29. The method for predicting the likelihood of developing gastric cancer of claim 20, wherein the preparation for measuring the protein level is an antibody or an aptamer that is specific to the protein.

30. A method for predicting the likelihood of developing metachronous gastric cancer, comprising:
(a) manufacturing a preparation for measuring the level of mRNA of any one or more genes selected from the group consisting of CDK1, KDF1, CREB5, and AKT2, or the level of proteins expressed therefrom;
(b) measuring the level of mRNA of any one or more genes selected from the group consisting of CDK1, KDF1, CREB5, and AKT2, or the level of proteins expressed therefrom in a biological sample isolated from a subject using the preparation; and
(c) comparing the measured mRNA or protein level to the level measured in a biological sample isolated from a control group.

31. A method for predicting the prognosis of early gastric cancer patients after treatment by endoscopic submucosal dissection, comprising:
(a) manufacturing a preparation for measuring the level of mRNA of any one or more genes selected from the group consisting of CDK1, KDF1, CREB5, and AKT2, or the level of proteins expressed therefrom
(b) measuring the level of mRNA of any one or more genes selected from the group consisting of CDK1, KDF1, CREB5, and AKT2, or the level of proteins expressed therefrom in a biological sample isolated from early gastric cancer patients using the preparation; and
(c) comparing the measured mRNA or protein level to the level measured in a biological sample isolated from a control group.
